SEQUENCE LISTING (11 sequences)

SEQ ID NO: 1 - RNA, 1265 nt, Bacillus velezensis
cgaugcguagccgaccugagagggugaucggccacacugggacugagacacggcccagacuccuacgggaggcagcaguagggaaucuuccgcaauggacgaaagucugacggagcaacgccgcgugagugaugaagguuuucggaucguaaagcucuguuguuagggaagaacaagugccguucaaauagggcggcaccuugacgguaccuaaccagaaagccacggcuaacuacgugccagcagccgcgguaauacguagguggcaagcguuguccggaauuauugggcguaaagggcucgcaggcgguuucuuaagucugaugugaaagcccccggcucaaccggggagggucauuggaaacuggggaacuugagugcagaagaggagaguggaauuccacguguagcggugaaaugcguagagauguggaggaacaccaguggcgaaggcgacucucuggucuguaacugacgcugaggagcgaaagcguggggagcgaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacgggggcccgcacaagcgguggagcaugugguuuaauucgaagcaacgcgaagaaccuuaccaggucuugacauccucugacaauccuagagauaggacguccccuucgggggcagagugacagguggugcaugguugucgucagcucgugucgugagauguuggguuaagucccgcaacgagcgcaacccuugaucuuaguugccagcauucaguugggcacucuaaggugacugccggugacaaaccggaggaagguggggaugacgucaaaucaucaugccccuuaugaccugggcuacacacgugcuacaauggacagaacaaagggcagcgaaaccgcgagguuaagccaaucccacaaaucuguucucaguucggaucgcagucugcaacucgacugcgugaagcuggaaucgcuaguaaucgcggaucagcaugccgcggugaauacguucccgggccuuguacacaccgcccgucacaccacgagaguuuguaacacccgaagucggugagguaaccuuuuaggagccagccgccgaaggugggacagaugauuggggugaagucguaacaagguagccguaucggaaggugcggcuggaucaccuccuuu

SEQ ID NO: 2 - RNA, 1265 nt, Bacillus velezensis; identical to SEQ ID NO: 1

SEQ ID NO: 3 - RNA, 1265 nt, Bacillus velezensis; identical to SEQ ID NO: 1

SEQ ID NO: 4 - DNA, 22 bp, Artificial sequence
  synthesized sequence
ctaaatatcggggagagaccga

SEQ ID NO: 5 - DNA, 29 bp, Artificial sequence
  synthesized sequence
gtacttttaactctctttccaaagtgctt

SEQ ID NO: 6 - DNA, 21 bp, Artificial sequence
  synthesized sequence
ttcatctttccctcacggtac

SEQ ID NO: 7 - DNA, 21 bp, Artificial sequence
  synthesized sequence
acaccattcccccctatctgt

SEQ ID NO: 8 - DNA, 21 bp, Artificial sequence
  synthesized sequence
tgaccaccagcatctgttgaa

SEQ ID NO: 9 - DNA, 19 bp, Artificial sequence
  synthesized sequence
tgccggcgcaaaacacctg

SEQ ID NO: 10 - DNA, 21 bp, Artificial sequence
  synthesized sequence
agagtttgatymtcctggctc

SEQ ID NO: 11 - DNA, 20 bp, Artificial sequence
  synthesized sequence
cggttaccttgttacgactt

(19) United States
(12) Patent Application Publication
(10) Pub. No.: US 2024/0277782 A1
(43) Pub. Date: Aug. 22, 2024

(54) COMPOSITIONS AND METHODS FOR MICROBIAL TREATMENT OF SKIN DISORDERS

(71) Applicant: IFF US Holding, LLC, Wilmington, DE (US)

(72) Inventors: Helene Flannery, Swedesboro, NJ (US); John Thomas Gannon, Hockessin, DE (US); Seung-Pyo Hong, Hockessin, DE (US); Rick W Ye, Wilmington, DE (US)

(21) Appl. No.: 18/569,824

(22) PCT Filed: Jun. 15, 2022
(86) PCT No.: PCT/US2022/033548

§ 371 Date: Dec. 13, 2023

Related U.S. Application Data
(60) Provisional application No. 63/216,660, filed on Jun. 30, 2021.

(51) Int. Cl. A61K 35/742; A61K 36/064; A61P 31/10; C12N 1/16; C12N 1/20; C12R 1/07; C12R 1/645

(52) U.S. Cl. CPC A61K 35/742; A61K 36/064; A61P 31/10; C12N 1/165; C12N 1/205; C12R 2001/07; C12R 2001/645

(57) ABSTRACT

The present disclosure is directed towards skin care compositions and methods for providing treatment of scalp disorders. More specifically, the present disclosure is directed towards methods and compositions comprising at least one microorganism of the genus Yarrowia, and/or a cell lysate thereof, and/or a fermentate thereof, and/or a metabolite thereof in combination with at least one Bacillus velezensis, and/or a cell lysate thereof, and/or fermentate thereof, and/or a metabolite thereof, for treating a scalp disorder, including dandruff.

### CROSS REFERENCE

This application is a 371 of International Application No. PCT/US2022/033548, filed Jun. 15, 2022, and claims the benefit of U.S. Provisional Application No. 63/216,660, filed Jun. 30, 2021, each of which is incorporated by reference in its entirety.

### FIELD OF THE DISCLOSURE

The present disclosure is directed towards skin care compositions and methods for providing treatment of scalp disorders. More specifically, the present disclosure is directed towards methods and compositions comprising at least one microorganism of the genus Yarrowia, and/or a cell lysate thereof, and/or a fermentate thereof, and/or a metabolite thereof in combination with at least one Bacillus velezensis, and/or a cell lysate thereof, and/or fermentate thereof, and/or a metabolite thereof, for treating a scalp disorder, including dandruff.

### REFERENCE TO SEQUENCE LISTING SUBMITTED ELECTRONICALLY

The content of the sequence listing electronically submitted with the application as an ASCII text file (20231208_NB41996USPCT_ST25; Size: 8192 bytes; Created: Dec. 8, 2023) forms part of the application and is hereby incorporated herein by reference in its entirety.

### BACKGROUND

The skin functions as a barrier protecting the organism from drying out as well as protecting the organism against the penetration of external, often harmful, substances.

The skin is also home to a diverse population of microbes, the majority of which are commensal (nonpathogenic permanent residents) or transient (temporary residents) organisms. In pathogenic interactions, only the microbe benefits, while the host is eventually harmed. Many skin pathogens can be typically found living on the skin as commensal organisms, but microbial dysbiosis (or microbial imbalance), host genetic variation, and immune status may drive the transition from commensal to pathogen (Findley, K. and Grice, E. A., The Skin Microbiome: A Focus on Pathogens and Their Association with Skin Disease. PLOS Pathog. 2014, 10).

The epidermis constitutes the outermost region of the skin tissue and as such forms the actual protective sheath against the environment. The outer layer of the epidermis (Stratum corneum or Horny layer) is the part which is in contact with the environment and the particular structure of the horny layer protects the skin as well as stabilizes its own flexibility by binding a defined amount of water (P. M. Elias, Structure and Function of the Stratum Corneum Permeability Barrier, Drug Dev. Res. 13, 1988, 97-105).

Spatially, the skin microbiota may extend to subepidermal compartments (Nakatsuji, T et al., The Microbiome Extends to Subepidermal Compartments of Normal Skin. Nat. Commun. 2013, 4). Regions such as the face, chest, and back, areas with a high density of sebaceous glands, promote growth of lipophilic microorganisms such as Propionibacterium and Malassezia.

Skin is a unique environment where microbes often exist as biofilms (Brandwein, et al., 2016. NPJ Biofilms Microbiomes 2:3). The biofilms can form on the epithelial surfaces of the skin or inside the follicles. In addition to cells, a biofilm consists of extracellular components such as exopolysaccharides, proteins, and DNA. This complex structure can be a physical and chemical barrier for certain compounds. But more importantly, the physiology of the microbes in the state of biofilm is very different than those in planktonic state. This is especially true for their ability to counter environmental stress and to resist various antimicrobial treatments (Koo, et al., Nature Reviews Microbiology 15:740-755, 2017).

Yeast Malassezia species isolated from both healthy and unhealthy skin have been shown to form biofilms in vitro (Angiolella, et al. 2020, Med Mycol. 0:1-7). These isolates of Malassezia globosa (M. globosa) can be highly adherent and/or hydrophobic as well as biofilm producers. Malassezia species in the form of biofilm have been shown to have a significant decrease in their susceptibility to antifungal agents (Figueredo, et al., 2016, Medical Mycology 8:863-867, 2013; Bumroogthai, et al., Medical Mycology 54:544-549). Biofilm adherence and hydrophobicity was suggested as virulence factor for Malassezia (Allen, et al., 2018, J. of Clinical & Experimental Dermatology Research 6:311, 2015; Angiolella, et al., Medical Mycology 56:110-116). Thus, strategies to remove Malassezia biofilm can be beneficial to treat various skin conditions caused by this group of organisms.

Malassezia is a predominant fungus of the skin microbiota and found on virtually everybody's scalp and implicated in the most common skin disorders such as, but not limiting to, seborrheic dermatitis, dandruff, and tinea versicolor. Dandruff is the common term for seborrhea of the scalp. It is mainly associated with Malassezia restricta (M. restricta) and Malassezia globosa (M. globosa) and has a very high prevalence of nearly 50% of the population (Schommer, N. N.; Gallo, R. L., Structure and Function of the Human Skin Microbiome. Trends Microbiol. 2013, 21, 660-668). Improvements in the disease can be achieved by therapeutic application of antifungal, but not antibacterial agents. The mechanisms underlying pathogenicity are incompletely understood. Impaired skin barrier function facilitates the course of the disease (Harding, C. R et al., Dandruff: a condition characterized by decreased levels of intercellular lipids in scalp stratum corneum and impaired barrier function. Arch. Dermatol. Res. 2002, 294, 221-230).

Malassezia species (Malassezia spp.) do not have fatty acid synthase, so they have to rely on sebum lipids for carbon source. They also lack delta 2,3-enoyl-CoA isomerase for efficient unsaturated FA (e.g. oleate) utilization. Malassezia species feeds on sebum fat (by secreting a lipase or lipases that splits triglycerides into irritant fatty acids), and as sebum fat is broken down, free fatty acids (such as oleic acid) are released as by-product. Many people are sensitive to free fatty acids as they can induce hyperproliferation and scaling, or induce the release of arachidonic acid, which is also involved in inflammation, and their scalp responds by becoming irritated. In respond to the irritation, the scalp starts to become inflamed, red, and itchy, and the body shed skin cells faster than usual, in attempt to shed the irritant. The shedding of skin causes visible flakes to appear on the scalp, which is dandruff.

In addition to yeast Malassesia species, Staphylococcus species, such as S. aureus or S. epidermidis, are associated with dandruff scalp while Propionibacterial species are associated with healthy scalp (Saxena, et al., 2018, Front Cell Infect Microbiol, 8:346; Xu, et al. 2016, Sci Rep., 6: 24877. Suppressing certain populations of Staphylococcus may provide benefit for dandruff treatment. Certain Staphylococcus species including S. aureus can form biofilms (Crit Rev Microbiol, 43:602-620, 2017).

There remains a need to find methods and skin care compositions for providing treatment of scalp disorders, such as but not limiting to methods and compositions for the treatment of a dandruff condition of the scalp.

### SUMMARY

The present disclosure is directed to compositions and methods for providing treatment of scalp disorders. More specifically, the present disclosure is directed towards methods and compositions comprising at least one microorganism of the genus Yarrowia, and/or a cell lysate thereof, and/or a fermentate thereof, and/or a metabolite thereof, in combination with at least one Bacillus velezensis, and/or a cell lysate thereof, and/or fermentate thereof, and/or a metabolite thereof, for treating a scalp disorder.

The present invention is based on the discovery that a fermentate mixture comprising a Bacillus velezensis fermentate supernatant and a Yarrowia lypolytica fermentate supernatant reduces the growth of Malassezia species and prevent or reduce the biofilm formation of Malassezia species.

The inventors have unexpectedly observed that a fermentate supernatant of Yarrowia can reduce the growth of Malassezia species and prevent or reduce the biofilm formation of Malassezia species. Furthermore, the inventors have unexpectedly observed that a fermentate supernatant of Bacillus velezensis reduces the growth of Malassezia species and prevent or reduce the biofilm formation of Malassezia species.

Unexpectedly, when the fermentate supernatants of Yarrowia lypolytica and Bacillus velezensis were combined and tested for growth inhibition against Malassezia species, an additive effect in the growth reduction of Malassezia species was observed. When the fermentate supernatants of Yarrowia lypolytica and Bacillus velezensis were combined and tested for biofilm formation of Malassezia species, an additive effect in the prevention and reduction of biofilm formation of Malassezia species was observed.

Together these observations indicate the unexpected effect that active components of the fermentate mixture comprising a combination of Yarrowia fermentate supernatant and Bacillus velezensis fermentate supernatant do not interfere with each other (do not inhibit or cancel each other's effect against Malassezia growth) but rather add each other's effects in a manner to form a fermentate supernatant mixture that can be used in compositions and methods for treating a scalp disorder, such as but not limiting to dandruff.

Furthermore, the inventors have unexpectedly observed that when a cell lysate of Yarrowia lypolytica and a cell lysate of Bacillus velezensis were combined and tested for growth inhibition against Malassezia species, an additive effect in the growth reduction of Malassezia species was observed, indicating that the cell lysate mixture can be used in compositions and methods for treating a scalp disorder.

In one aspect the fermentate supernatants of Yarrowia lypolytica and/or Bacillus velezensis and cell lysates of Yarrowia lypolytica and/or Bacillus velezensis can be combined and used as an active ingredient in a skin care composition wherein said composition reduces and/or treats said scalp disorder.

In one embodiment, the composition is a skin care composition for use in the treatment of a scalp disorder, comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder.

In one aspect the scalp disorder is selected from the group consisting of a dandruff condition of the scalp, unbalanced ecoflora of the scalp, discomfort of the scalp, tinea versicolor, dry skin, irritated skin, seborrheic dermatitis, and any one combination thereof.

In one embodiment, the composition is a skin care composition for use in the treatment of a scalp disorder, comprising a fermentate of at least two microorganisms selected from the group consisting of Yarrowia lipolytica and Bacillus velezensis, wherein said composition reduces and/or treats said scalp disorder, wherein the fermentate is a fermentate supernatant mixture comprising a fermentate supernatant of Yarrowia lipolytica combined with a fermentate supernatant of Bacillus velezensis.

In one aspect, the skin care composition comprises a fermentate supernatant of Yarrowia lipolytica (YSP) at about 0.01% to 10% by weight and a fermentate supernatant of Bacillus velezensis (BSP) at about 0.01% to 10% by weight, relative to a total weight of said skin care composition.

In one embodiment, the composition is a skin care composition for use in the treatment of a scalp disorder, comprising at least two microorganisms selected from the group consisting of Yarrowia lipolytica and Bacillus velezensis, and/or cell lysate thereof, and/or fermentate thereof, and/or metabolite thereof, wherein said composition reduces the growth of Malassezia species, and/or prevents or reduces biofilm formation of Malassezia species.

In one embodiment, the composition is a fermentate mixture comprising a fermentate of Yarrowia lipolytica combined with a fermentate from Bacillus velezensis for use in treatment of a scalp disorder.

In one embodiment, the method is a method for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition.

In one embodiment, the method is a method for treating and/or reducing a dandruff condition of the scalp in a subject in need thereof, comprising administering a skin care composition comprising a fermentate from at least two microorganisms selected from the group consisting of Yarrowia lipolytica and Bacillus velezensis, wherein said composition reduces the growth of Malassezia species, and/or prevents or reduces biofilm formation of Malassezia species and/or reduces and/or treats said dandruff condition.

In one embodiment, the method is a method for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition.

### DETAILED DESCRIPTION

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. It will be understood that in the following, embodiments referred to in relation to one broad aspect of the invention are equally applicable to each of the other broad aspects of the present invention described above. It will be further understood that, unless the context dictates otherwise, the embodiments described below may be combined.

### Scalp Disorders

For the purpose of the present disclosure, the term “scalp disorder” includes a dandruff condition of the scalp, unbalanced ecoflora of the scalp, discomfort of the scalp, tinea versicolor, dry skin, irritated skin, seborrheic dermatitis, or any one combination thereof.

For the purpose of the present disclosure, the term “dandruff condition” refers to a condition manifested by a scalp presenting excessive dryness or excessive secretion of sebum, which, depending on the case, may be characterized by the presence of dry or greasy or oily dandruff, or even pruritis and/or an inflammation of the epidermis.

Dry dandruff conditions reflect a xerosis of the scalp, which may be combined with excessively rapid renewal of its stratum corneum. Dry dandruff flakes are generally in the form of small white or grey flakes and are spread over the scalp and on the clothing, giving rise to an unaesthetic visual effect.

The itching associated with dryness of the scalp may lead to erythema, pruritus or even inflammation.

Greasy or oily dandruff conditions are one of the forms of seborrheic dermatitis. Individuals suffering from seborrheic dermatitis have an erythematous scalp covered with large, greasy or oily, yellow scales which accumulate so as to form packets. They have a pruritic scalp, and often have burning sensations on the affected areas. These phenomena may be amplified by the presence of pathogenic microorganisms, especially Malassezia species (Malassezia spp.). Malassezia species described herein include, but are not limited to, Malassezia restricta (M. restricta) and Malassezia globosa (M. globosa). These microorganisms having the property of releasing fatty acids from the sebum may impair the barrier function of the epidermis and give rise to inflammation.

During dandruff conditions of the scalp, the cutaneous barrier is unbalanced, its integrity and its hydration are impaired, and its ecoflora is disturbed. The skin of the scalp is irritated and pruritic, brittle, less hydrated, and sensitive to infections.

In addition to yeast Malassesia species, Staphylococcus species, such as S. aureus or S. epidermidis, are associated with dandruff scalp while Propionibacterial species are associated with healthy scalp (Saxena, et al., 2018, Front Cell Infect Microbiol, 8:346; Xu, et al. 2016, Sci Rep., 6: 24877. Suppressing certain populations of Staphylococcus may provide benefit for dandruff treatment. Certain Staphylococcus species including S. aureus can form biofilms (Crit Rev Microbiol, 43:602-620, 2017).

Malassezia species described herein include, but are not limited to, Malassezia restricta (M. restricta), Malassezia globosa (M. globosa), Malassezia furfur (M. furfur), Malassezia sympodialis (M. sympodialis), Malassezia phylotype 5 (M. phylotype 5), other uncharacterized Malassezia species, and any combination thereof.

Staphylococcus species described herein include, but are not limited to, Staphylococcus aureus, Staphylococcus epidermidis, Staphylococcus haemolyticus, Staphylococcus capitis, other uncharacterized Staphylococcus species, and any combination thereof.

Unexpectedly, the inventors have observed that a fermentate mixture comprising a Bacillus velezensis fermentate supernatant and a Yarrowia lypolytica fermentate supernatant, reduces the growth of Malassezia species and prevent or reduce the biofilm formation of Malassezia species. Furthermore, the inventors have unexpectedly observed that when a cell lysate of Yarrowia lypolytica and a cell lysate of Bacillus velezensis were combined and tested for growth inhibition against Malassezia species, an additive effect in the growth reduction of Malassezia species was observed.

The use of a microorganism, in particular of Yarrowia, in accordance with the disclosure can result in the reduction of free fatty acids produced by lipid degrading activities of the dandruff inducing Malassezia species, and M. globosa in particular, thereby making it possible to reduce dandruff conditions in subjects in need thereof. Fatty acids might be produced from other lipophilic bacteria. The reduction in occurrence of fatty acids can reduce dandruff and other skin disorders caused by free fatty acids

This decrease can be reflected by a reduction in the phases of scratching the scalp and a reduction in the impairment of the barrier function resulting therefrom. The skin is then less irritated and less pruritic, and the presence of the dandruff is reduced, or even eliminated.

### Microorganisms, Cell-Lysates, Fermentates, Metabolites and Fractions Thereof.

As used herein, “microorganism” or “microbe” refers to a bacterium, a fungus, a virus, a protozoan, archaea, and other microbes or microscopic organisms.

In some embodiments, the microorganism(s) suitable for use in the present invention can be subjected to treatments that render them non-replicating, for example, exposure to heat, desiccation, γ-irradiation, or UV-irradiation. A non-replicating microorganism(s) suitable for use in the present invention can be a dead cell or a living cell that has been rendered incapable of cell division. A non-replicating microorganism(s) suitable for use in the present invention can be an intact cell or a cell that has undergone partial or complete lysis. In some embodiments, the non-replicating cells can include a mixture of intact and lysed cells.

The microorganism(s) suitable for use in the present invention may be included in a composition according to the invention in live, semi-active or inactivated or dead form. For the purposes of the invention, an “inactivated” or “dead” microorganism is a microorganism that is no longer capable of forming colonies in cultures. The dead or inactivated microorganisms may have intact or broken cell membranes. The dead or inactivated microorganisms may be obtained via any method known to those skilled in the art.

In one aspect the microorganisms suitable for use in the present invention includes the microorganism of the genus Yarrowia, the microorganism Bacillus velezensis, and any one combination thereof.

In one aspect, the microorganism of the genus Yarrowia is at least one microorganism selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof.

In some aspects, the microorganism suitable for use in the present invention includes a bacterial strain having a 16S ribosomal RNA sequence displaying at least 97.0% sequence similarity to a 16S ribosomal RNA sequence of Bacillus velezensis E04 (SEQ ID NO: 1) deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 (SEQ ID NO: 2) deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 (SEQ ID NO: 3) deposited at Westerdijk Fungal Biodiversity Institute (WFDB) under number CBS147471, and any one combination thereof.

The 16S ribosomal RNA sequence of Bacillus velezensis E04 is as follows:

(SEQ ID NO: 1)
CGAUGCGUAGCCGACCUGAGAGGGUGAUCGGCCACACUGGGACUGA
GACACGGCCCAGACUCCUACGGGAGGCAGCAGUAGGGAAUCUUCCGCAAU
GGACGAAAGUCUGACGGAGCAACGCCGCGUGAGUGAUGAAGGUUUUCGGA
UCGUAAAGCUCUGUUGUUAGGGAAGAACAAGUGCCGUUCAAAUAGGGCGG
CACCUUGACGGUACCUAACCAGAAAGCCACGGCUAACUACGUGCCAGCAG
CCGCGGUAAUACGUAGGUGGCAAGCGUUGUCCGGAAUUAUUGGGCGUAAA
GGGCUCGCAGGCGGUUUCUUAAGUCUGAUGUGAAAGCCCCCGGCUCAACC
GGGGAGGGUCAUUGGAAACUGGGGAACUUGAGUGCAGAAGAGGAGAGUGG
AAUUCCACGUGUAGCGGUGAAAUGCGUAGAGAUGUGGAGGAACACCAGUG
GCGAAGGCGACUCUCUGGUCUGUAACUGACGCUGAGGAGCGAAAGCGUGG
GGAGCGAACAGGAUUAGAUACCCUGGUAGUCCACGCCGUAAACGAUGAGU
GCUAAGUGUUAGGGGGUUUCCGCCCCUUAGUGCUGCAGCUAACGCAUUAA
GCACUCCGCCUGGGGAGUACGGUCGCAAGACUGAAACUCAAAGGAAUUGA
CGGGGGCCCGCACAAGCGGUGGAGCAUGUGGUUUAAUUCGAAGCAACGCG
AAGAACCUUACCAGGUCUUGACAUCCUCUGACAAUCCUAGAGAUAGGACG
UCCCCUUCGGGGGCAGAGUGACAGGUGGUGCAUGGUUGUCGUCAGCUCGU
GUCGUGAGAUGUUGGGUUAAGUCCCGCAACGAGCGCAACCCUUGAUCUUA
GUUGCCAGCAUUCAGUUGGGCACUCUAAGGUGACUGCCGGUGACAAACCG
GAGGAAGGUGGGGAUGACGUCAAAUCAUCAUGCCCCUUAUGACCUGGGCU
ACACACGUGCUACAAUGGACAGAACAAAGGGCAGCGAAACCGCGAGGUUA
AGCCAAUCCCACAAAUCUGUUCUCAGUUCGGAUCGCAGUCUGCAACUCGA
CUGCGUGAAGCUGGAAUCGCUAGUAAUCGCGGAUCAGCAUGCCGCGGUGA
AUACGUUCCCGGGCCUUGUACACACCGCCCGUCACACCACGAGAGUUUGU
AACACCCGAAGUCGGUGAGGUAACCUUUUAGGAGCCAGCCGCCGAAGGUG
GGACAGAUGAUUGGGGUGAAGUCGUAACAAGGUAGCCGUAUCGGAAGGUG
CGGCUGGAUCACCUCCUUU.

The 16S ribosomal RNA sequence of Bacillus velezensis H02 is as follows:

(SEQ ID NO: 2)
CGAUGCGUAGCCGACCUGAGAGGGUGAUCGGCCACACUGGGACUGA
GACACGGCCCAGACUCCUACGGGAGGCAGCAGUAGGGAAUCUUCCGCAAU
GGACGAAAGUCUGACGGAGCAACGCCGCGUGAGUGAUGAAGGUUUUCGGA
UCGUAAAGCUCUGUUGUUAGGGAAGAACAAGUGCCGUUCAAAUAGGGCGG
CACCUUGACGGUACCUAACCAGAAAGCCACGGCUAACUACGUGCCAGCAG
CCGCGGUAAUACGUAGGUGGCAAGCGUUGUCCGGAAUUAUUGGGCGUAAA
GGGCUCGCAGGCGGUUUCUUAAGUCUGAUGUGAAAGCCCCCGGCUCAACC
GGGGAGGGUCAUUGGAAACUGGGGAACUUGAGUGCAGAAGAGGAGAGUGG
AAUUCCACGUGUAGCGGUGAAAUGCGUAGAGAUGUGGAGGAACACCAGUG
GCGAAGGCGACUCUCUGGUCUGUAACUGACGCUGAGGAGCGAAAGCGUGG
GGAGCGAACAGGAUUAGAUACCCUGGUAGUCCACGCCGUAAACGAUGAGU
GCUAAGUGUUAGGGGGUUUCCGCCCCUUAGUGCUGCAGCUAACGCAUUAA
GCACUCCGCCUGGGGAGUACGGUCGCAAGACUGAAACUCAAAGGAAUUGA
CGGGGGCCCGCACAAGCGGUGGAGCAUGUGGUUUAAUUCGAAGCAACGCG
AAGAACCUUACCAGGUCUUGACAUCCUCUGACAAUCCUAGAGAUAGGACG
UCCCCUUCGGGGGCAGAGUGACAGGUGGUGCAUGGUUGUCGUCAGCUCGU
GUCGUGAGAUGUUGGGUUAAGUCCCGCAACGAGCGCAACCCUUGAUCUUA
GUUGCCAGCAUUCAGUUGGGCACUCUAAGGUGACUGCCGGUGACAAACCG
GAGGAAGGUGGGGAUGACGUCAAAUCAUCAUGCCCCUUAUGACCUGGGCU
ACACACGUGCUACAAUGGACAGAACAAAGGGCAGCGAAACCGCGAGGUUA
AGCCAAUCCCACAAAUCUGUUCUCAGUUCGGAUCGCAGUCUGCAACUCGA
CUGCGUGAAGCUGGAAUCGCUAGUAAUCGCGGAUCAGCAUGCCGCGGUGA
AUACGUUCCCGGGCCUUGUACACACCGCCCGUCACACCACGAGAGUUUGU
AACACCCGAAGUCGGUGAGGUAACCUUUUAGGAGCCAGCCGCCGAAGGUG
GGACAGAUGAUUGGGGUGAAGUCGUAACAAGGUAGCCGUAUCGGAAGGUG
CGGCUGGAUCACCUCCUUU.

The 16S ribosomal RNA sequence of Bacillus velezensis F03 is as follows:

(SEQ ID NO: 3)
CGAUGCGUAGCCGACCUGAGAGGGUGAUCGGCCACACUGGGACUGAGACA
CGGCCCAGACUCCUACGGGAGGCAGCAGUAGGGAAUCUUCCGCAAUGGAC
GAAAGUCUGACGGAGCAACGCCGCGUGAGUGAUGAAGGUUUUCGGAUCGU
AAAGCUCUGUUGUUAGGGAAGAACAAGUGCCGUUCAAAUAGGGCGGCACC
UUGACGGUACCUAACCAGAAAGCCACGGCUAACUACGUGCCAGCAGCCGC
GGUAAUACGUAGGUGGCAAGCGUUGUCCGGAAUUAUUGGGCGUAAAGGGC
UCGCAGGCGGUUUCUUAAGUCUGAUGUGAAAGCCCCCGGCUCAACCGGGG
AGGGUCAUUGGAAACUGGGGAACUUGAGUGCAGAAGAGGAGAGUGGAAUU
CCACGUGUAGCGGUGAAAUGCGUAGAGAUGUGGAGGAACACCAGUGGCGA
AGGCGACUCUCUGGUCUGUAACUGACGCUGAGGAGCGAAAGCGUGGGGAG
CGAACAGGAUUAGAUACCCUGGUAGUCCACGCCGUAAACGAUGAGUGCUA
AGUGUUAGGGGGUUUCCGCCCCUUAGUGCUGCAGCUAACGCAUUAAGCAC
UCCGCCUGGGGAGUACGGUCGCAAGACUGAAACUCAAAGGAAUUGACGGG
GGCCCGCACAAGCGGUGGAGCAUGUGGUUUAAUUCGAAGCAACGCGAAGA
ACCUUACCAGGUCUUGACAUCCUCUGACAAUCCUAGAGAUAGGACGUCCC
CUUCGGGGGCAGAGUGACAGGUGGUGCAUGGUUGUCGUCAGCUCGUGUCG
UGAGAUGUUGGGUUAAGUCCCGCAACGAGCGCAACCCUUGAUCUUAGUUG
CCAGCAUUCAGUUGGGCACUCUAAGGUGACUGCCGGUGACAAACCGGAGG
AAGGUGGGGAUGACGUCAAAUCAUCAUGCCCCUUAUGACCUGGGCUACAC
ACGUGCUACAAUGGACAGAACAAAGGGCAGCGAAACCGCGAGGUUAAGCC
AAUCCCACAAAUCUGUUCUCAGUUCGGAUCGCAGUCUGCAACUCGACUGC
GUGAAGCUGGAAUCGCUAGUAAUCGCGGAUCAGCAUGCCGCGGUGAAUAC
GUUCCCGGGCCUUGUACACACCGCCCGUCACACCACGAGAGUUUGUAACA
CCCGAAGUCGGUGAGGUAACCUUUUAGGAGCCAGCCGCCGAAGGUGGGAC
AGAUGAUUGGGGUGAAGUCGUAACAAGGUAGCCGUAUCGGAAGGUGCGGC
UGGAUCACCUCCUUU.

In one aspect of the invention, cell lysates are provided.

As used herein, the term “cell lysate” or “lysate” refers to microbial cells which have been lysed by any suitable means. The term “cell lysate” or “lysate” conventionally denotes a material obtained after the destruction or dissolution of biological cells via a phenomenon known as cell lysis, thus giving rise to the release of the intracellular biological constituents naturally contained in the cells of the microorganism under consideration. For the purposes of the present disclosure, the term “lysate” is used without preference to denote the whole lysate obtained via lysis of the microorganism under consideration or only a fraction thereof. The lysate used is thus totally or partially formed from the intracellular biological constituents and from the constituents of the cell walls and membranes. A lysate used for the invention may be the whole lysate obtained via lysis of the microorganism under consideration, or a fraction thereof. This cell lysis may be accomplished by any suitable means or any one method known in the art, such as but not limiting to, an osmotic shock, a heat shock, ultrasonication, sonication, homogenization, shearing, chemical lysis or under a mechanical stress of centrifugation type. In some embodiments, the cell debris is removed from the cell lysate prior to use. In some embodiments the cell lysates are filtered or fractionated prior to use.

Cell lysates for use in the present invention include cell lysates from the microorganism of the genus Yarrowia, the microorganism Bacillus velezensis, and any one combination thereof.

As described herein, when a cell lysate of Yarrowia lypolytica and a cell lysate of Bacillus velezensis were combined and tested for growth inhibition against Malassezia species, an additive effect in the growth reduction of Malassezia species was observed, indicating that said cell lysate mixture can be used in compositions and methods for treating a scalp disorder.

In one aspect the fermentate supernatants of Yarrowia lypolytica and/or Bacillus velezensis and the cell lysates of Yarrowia lypolytica and/or Bacillus velezensis can be combined and used as an active ingredient in a skin care composition wherein said composition reduces and/or treats said scalp disorder.

In one aspect the cell lysate for use in the present invention includes a cell lysate from the microorganism of the genus Yarrowia selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof.

In one aspect the cell lysate for use in the present invention includes a cell lysates from Bacillus velezensis, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one aspect, the cell lysate is a cell lysate of the microorganism of the genus Yarrowia, wherein said cell lysate comprises one or more active compounds (also referred to as skin care benefit agents) derived from the culture and metabolism of a Yarrowia microorganism, and also having efficacy in the treatment of a scalp disorder.

In one aspect, the cell lysate is a cell lysate of the microorganism Bacillus velezensis, wherein said cell lysate comprises one or more active compounds (also referred to as skin care benefit agents) derived from the culture and metabolism of a Bacillus velezensis, and also having efficacy in the treatment of a scalp disorder.

In one aspect, the cell lysate is a mixture of the cell lysate of the microorganism of the genus Yarrowia with the cell lysate of Bacillus velezensis, wherein said cell lysate mixture comprises one or more active compounds (also referred to as skin care benefit agents) derived from the culture and metabolism of a Yarrowia microorganism and the culture and metabolism of a Bacillus velezensis and also having efficacy in the treatment of a scalp disorder.

It will be apparent that the cell lysate may be used directly in the compositions and methods of the present invention, or that one or more of the actives (such as but not limiting to metabolites) may be isolated form the cell lysate by any suitable means prior to use.

A lysate may be used in various forms, in the form of a solution or in a pulverulent form.

In one aspect of the invention, fermentates are provided.

As used herein, the term “fermentate” is to be understood as a composition (complex mixture) produced by propagating living microorganisms (microbial strains) in a nutrient medium. The fermentate may include a cellular mass component from said microorganisms, unspent media components, and metabolites (i.e., unused substrates and/or fermentation end-products). As used herein, a “cellular mass component” refers to any mixture of proteins, lipids (i.e., membranes), carbohydrates, metabolites, etc. from the propagated microorganism. For example, as a microorganism grows it produces new cells that generally include additional cellular mass such as, without limitation, cell membranes, nucleic acids (i.e., DNA and/or RNA) internal subcellular structures, and proteins (i.e., membrane-bound, secreted, and/or intracellular).

Fermentates for use in the present invention include fermentates from the microorganism of the genus Yarrowia, the microorganism Bacillus velezensis, and/or any one combination thereof.

It will be apparent that the fermentate may be used directly in the compositions and methods of the present invention, or that one or more fractions of said fermentate comprising active ingredients (skin care benefit agents) may be isolated form the fermentate by any suitable means prior to use.

Fermentates can be further concentrated prior to be included in a skin care composition to obtain an effective amount of a skin care active in said fermentate. Fermentates can be spray-dried or lyophilized prior to be include in a skin care composition.

In one aspect the fermentate is a fermentate supernatant. As used herein, a “fermentate supernatant” or “cell free supernatant”, “fermentation supernatant” or “fermentate filtrate” are used interchangeably and refer to a fermentate that is substantially free of viable cells, such as a supernatant of a cell culture of at least one microorganism from which the cells have been removed. It is understood that cells can be removed from the cell culture by any method known in the art and that such removal of cells (such as through centrifugation, filtration) may still results in cell free supernatants that can comprise a trace amount of cells or cell debris.

In one embodiment the fermentate supernatant is obtained by filtration or centrifugation of the culture medium in which Yarrowia cells were cultivated.

In one embodiment the fermentate supernatant is obtained by filtration or centrifugation of the culture medium in which Bacillus velezensis cells were cultivated.

Fermentates, fermentate supernatants or fractions thereof for use in the present invention include fermentates, fermentate supernatants or fractions thereof from the microorganism of the genus Yarrowia, the microorganism Bacillus velezensis, and/or any one combination thereof.

In one aspect the fermentates, fermentate supernatants or fractions thereof for use in the present invention includes fermentates, fermentate supernatants or fractions thereof from the microorganism of the genus Yarrowia selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof . . .

In one aspect the fermentates, fermentate supernatants or fractions thereof for use in the present invention includes fermentates, fermentate supernatants or fractions thereof from the microorganism Bacillus velezensis, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one aspect, the fermentate, fermentate supernatant or fraction thereof, is a fermentate, fermentate supernatant or fraction thereof of the microorganism of the genus Yarrowia, wherein said fermentate, fermentate supernatant or fraction thereof comprises one or more active compounds (also referred to as skin care benefit agents) derived from the culture and metabolism of a Yarrowia microorganism, providing growth inhibition of dandruff inducing microorganism and/or having efficacy in the treatment of a scalp disorder.

In one aspect, the fermentate, fermentate supernatant or fraction thereof is a fermentate, fermentate supernatant or fraction thereof of the microorganism Bacillus velezensis, wherein said fermentate, fermentate supernatant or fraction thereof comprises one or more active compounds (also referred to as skin care benefit agents) derived from the culture and metabolism of a Bacillus velezensis, providing growth inhibition of dandruff inducing microorganism and/or having efficacy in the treatment of a scalp disorder.

In one aspect, the fermentate, fermentate supernatant or fraction thereof is a fermentate mixture, fermentate supernatant mixture of the fermentate, fermentate supernatant or fraction thereof of Yarrowia mixed with the fermentate, fermentate supernatant or fraction thereof of Bacillus velezensis, wherein said fermentate mixture comprises one or more active compounds (also referred to as skin care benefit agents) derived from the culture and metabolism of a Yarrowia microorganism a Bacillus velezensis, providing growth inhibition of dandruff inducing microorganism and/or having efficacy in the treatment of a scalp disorder.

As used herein, the term “fermentate mixture” refers to a combination of fermentates from at least two microorganisms selected from the group consisting of Yarrowia and Bacillus velezensis.

In one aspect the fermentate mixtures comprise fermentates of the genus Yarrowia selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, or any one combination thereof, combined with fermentates from the microorganism Bacillus velezensis, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

As used herein, the term “fermentate supernatant mixture” refers to a combination of fermentate supernatants from at least two microorganisms selected from the group consisting of Yarrowia and Bacillus velezensis.

In one aspect the fermentate supernatant mixtures comprise fermentate supernatants of the genus Yarrowia selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, or any one combination thereof, combined with fermentate supernatants from the microorganism Bacillus velezensis, wherein said Bacillus velezensis is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In some embodiments, the compositions of the invention can include fermentate mixtures (fermentate supernatant mixtures) of Yarrowia fermentates with Bacillus velezensis fermentates, from which all or substantially all, of the cells have been removed. Methods for separating cells from growth media are well known in the art and can rely upon physical methods, for example, centrifugation to produce a cell pellet and a culture supernatant, filtration, ultrafiltration, tangential flow-filtration, normal flow filtration or reverse osmosis. Alternatively, or in addition, the separation method can be ligand-based and include, for example, an antibody that specifically binds to Yarrowia and/or Bacillus velezensis. The antibody can be coupled to a solid support such as a magnetic bead.

In one embodiment the fermentate mixture for use in the present invention comprises Yarrowia fermentate mixed with Bacillus velezensis fermentate consisting essentially of cell free fermentate. The term “consisting essentially of” in the context of the fermentate includes that at least 90% of the fermentate have the indicated property (e.g. being cell free fermentate). Suitably at least 95% have the indicated property. Suitably at least 97% have the indicated property. Suitably at least 99% have the indicated property. In some embodiments at least 100% have the indicated property.

In one embodiment, the composition is a skin care composition for use in the treatment of a scalp disorder, comprising a fermentate of at least two microorganisms selected from the group consisting of Yarrowia lipolytica and Bacillus velezensis, wherein said composition reduces and/or treats said scalp disorder, wherein the fermentate is a fermentate supernatant mixture comprising a fermentate supernatant of Yarrowia lipolytica combined with a fermentate supernatant of Bacillus velezensis.

In one embodiment, the fermentate for use in the compositions and methods and/or uses of the present invention may be substantially free of viable Yarrowia and Bacillus velezensis cells, typically containing zero (or substantially zero) viable cells/mL fermentate.

In one aspect the fermentate, and/or fermentate fraction, and/or fermentate filtrate may comprise one or more metabolites, such as but not limiting to soluble metabolites, that were produced during the fermentation of at least one microorganism of the genus Yarrowia.

In one embodiment a fermentate originating from the culture (fermentation) of at least one microorganism of the genus Yarrowia in combination with a fermentate originating from the culture (fermentation) of Bacillus velezensis may be used in the compositions, methods and/or uses of the present invention.

The nutrient medium used for preparing the fermentate is any medium comprising necessary nutrients suitable for propagating the microorganism(s) suitable for use in the present invention. Suitable nutrients include but are not limited to amino peptides, peptides, yeast extract and/or vitamins. The medium can be based on dairy products, such as milk, cereals, fruits and/or vegetables.

As used herein, the term “metabolite(s) thereof” or “metabolite(s) of the microorganism(s) suitable for use in the present invention” or “metabolite actives” are used interchangeably and refer to any substance derived from the metabolism of a microorganism(s) suitable for use in the present invention and also having efficacy in the treatment of a scalp disorder.

In one aspect, the one or more metabolite(s) were produced during the culture (fermentation) of the least one microorganism of the genus Yarrowia, and/or the culture (fermentation) of Bacillus velezensis, for use in the treatment of a scalp disorder.

As used herein, the term “soluble metabolite” refers to a metabolite or metabolites present in the supernatant of a cell culture (fermentate supernatant) from which the cells have been removed. In one embodiment the culture is grown to a cell density of at least about OD600 0.5. In one embodiment the cells are removed by centrifugation. In one embodiment the supernatant is filtered. It will be apparent that the supernatant may be used directly in the formulations of the present invention, or that one or more of the metabolites may be isolated form the supernatant by any suitable means prior to use.

In some embodiments, the compositions of the invention can include Yarrowia fermentates, from which all or substantially all, of the Yarrowia cells have been removed. Methods for separating cells from growth media are well known in the art and can rely upon physical methods, for example, centrifugation to produce a cell pellet and a culture supernatant, filtration, ultrafiltration, tangential flow-filtration, normal flow filtration or reverse osmosis. Alternatively, or in addition, the separation method can be ligand-based and include, for example, an antibody that specifically binds to Yarrowia. The antibody can be coupled to a solid support such as a magnetic bead.

It will be apparent that fractions of the microorganism(s) suitable for use in the present invention, and/or fractions of cell lysates, and/or fractions of fermentates, and/or metabolites originating thereof, suitable for use in the present invention, may be used directly in the compositions and methods of the present invention, or that one or more of the actives (skin care benefit agents) may be isolated from said fractions by any suitable means prior to use.

### Skin Care Compositions for the Treatment of Skin Disorders Such as a Dandruff Condition.

As used herein the term “skin care composition(s)” refers to composition(s) comprising at least one skin care benefit agent (active agent) capable of providing a skin care benefit.

As used herein the term “skin care benefit agent” or “active agent” are used interchangeably, and refer to a microorganism, and/or a cell lysate of said microorganism, and/or a fermentate of said microorganism, and/or a metabolite of said microorganism that can provide a skin care benefit.

In one aspect, the microorganisms suitable for use in the present invention includes the microorganism of the genus Yarrowia, the microorganism Bacillus velezensis, and any one combination thereof.

In some aspect, the microorganism of the genus Yarrowia is at least one microorganism selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, and any one combination thereof.

In some aspects, the microorganism suitable for use in the present invention includes a bacterial strain having a 16S ribosomal RNA sequence displaying at least 97.0% sequence similarity to a 16S ribosomal RNA sequence of B. velezensis E04 (SEQ ID NO: 1) deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 (SEQ ID NO: 2) deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 (SEQ ID NO: 3) deposited at Westerdijk Fungal Biodiversity Institute (WFDB) under number CBS147471, and any one combination thereof.

As used herein the term “skin care benefit” refers to a benefit provided by an active agent (or skin care composition comprising an effective amount of said active agent) when applied topically to a skin.

In one aspect of the invention the skin care benefit is selected from the group consisting of preventing a dandruff condition, reducing a dandruff condition, treatment of a dandruff condition, reducing the growth of Malassezia species, reducing the occurrence of Malassezia species on the skin (scalp), removing biofilm formation of Malassezia species on the skin (scalp), preventing or reducing biofilm formation of Malassezia species on the skin (scalp), improving the barrier function of the skin, skin moisturizing (protecting the skin against dehydration by maintaining, restoring and/or strengthening the moisturization of the skin) or any one combination thereof.

As used herein, the term “biofilm” refers to a community of microorganisms embedded in an extracellular polymer matrix attached to a surface. The extracellular polymer matrix is a polymeric conglomeration generally composed of extracellular DNA, proteins, and polysaccharides. A biofilm may have one or more microorganisms and further includes water and may include other trapped particles. The microorganisms may be gram positive or gram-negative bacteria (aerobic or anaerobic); algae, protozoa, and/or yeast or filamentous fungi. In one embodiment, the biofilm is living cells including one or more Malassezia species.

As used herein, “surface” means any structure having sufficient mass to allow for attachment of biofilm. A surface includes a hard surface and a soft surface. Hard surfaces include, but are not limited to metal, glass, ceramics, wood, minerals (rock, stone, marble, granite), aggregate materials such as concrete, plastics, composite materials, hard rubber materials, and gypsum. Other surfaces may be biological surfaces, such as skin, scalp, or keratin.

Additional benefit agents for skin care can include antidandruff active agents.

Examples of antidandruff active agents include keratolytic agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones (e.g. ciclopirox), naturopathic agents such as Melaleuca sp. oil, Aloe vera, and probiotic microorganisms. (Indian J. Dermatol, 2010 April-June; 55(2): 130-134).

In one aspect, the skin care benefit agent (active agent) consists of at least a fermentate, fermentate supernatant or fraction thereof of the microorganism of the genus Yarrowia, wherein said fermentate, fermentate supernatant or fraction thereof comprises one or more active compounds derived from the culture and metabolism of a Yarrowia microorganism, providing growth inhibition of dandruff inducing microorganism and/or having efficacy in the treatment of a scalp disorder.

In one aspect, the skin care benefit agent (active agent) consists of at least a fermentate, fermentate supernatant or fraction thereof of the microorganism Bacillus velezensis, wherein said fermentate, fermentate supernatant or fraction thereof comprises one or more active derived from the culture and metabolism of a Bacillus velezensis, providing growth inhibition of dandruff inducing microorganism and/or having efficacy in the treatment of a scalp disorder.

In one aspect, the skin care benefit agent (active agent) consists of a fermentate mixture, fermentate supernatant mixture of the fermentate, fermentate supernatant or fraction thereof of Yarrowia mixed with the fermentate, fermentate supernatant or fraction thereof of Bacillus velezensis, wherein said fermentate mixture comprises one or more active compounds derived from the culture and metabolism of a Yarrowia microorganism and a Bacillus velezensis, providing growth inhibition of dandruff inducing microorganism and/or having efficacy in the treatment of a scalp disorder.

In one aspect, the skin care benefit agent (active agent) suitable for use in the present invention is formulated in a skin care composition.

It will be understood that the skin care composition for use in the present invention may comprise at least one microorganism of the genus Yarrowia, and/or at least one cell lysate of Yarrowia, and/or at least one fermentate of Yarrowia.

It will be understood that the skin care composition for use in the present invention may comprise at least one strain of Bacillus velezensis, and/or at least one cell lysate of Bacillus velezensis, and/or at least one fermentate of Bacillus velezensis.

In one embodiment, the composition is a skin care composition for use in the treatment of a scalp disorder, comprising a fermentate of at least two microorganisms selected from the group consisting of Yarrowia lipolytica and Bacillus velezensis, wherein said composition reduces and/or treats said scalp disorder, wherein the fermentate is a fermentate supernatant mixture comprising a fermentate supernatant of Yarrowia lipolytica combined with a fermentate supernatant of Bacillus velezensis.

It will be further apparent that the skin care composition for use according to the present invention may comprise, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0 about 45.0%, about 50.0% by weight of the microorganism(s), and/or a cell lysate thereof, and/or fermentate thereof, and/or metabolite thereof.

In one embodiment, the composition is a skin care composition for use in the treatment of a scalp disorder, comprising a fermentate of at least two microorganisms selected from the group consisting of Yarrowia lipolytica and Bacillus velezensis, wherein said composition reduces and/or treats said scalp disorder, wherein the fermentate is a fermentate supernatant mixture comprising a fermentate supernatant of Yarrowia lipolytica combined with a fermentate supernatant of Bacillus velezensis, wherein the fermentate supernatant of Yarrowia lipolytica (YSP) and the fermentate supernatant of Bacillus velezensis (BSP) are combined in a ratio of YSP: BSP from 10:1 to 1:10 by weight or by volume.

In one aspect the fermentate supernatant of Yarrowia lipolytica (YSP) and Bacillus velezensis (BSP) are combined in a ratio of YSP:BSP of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 10:2, 9:2, 8:2, 7:2, 6:2, 5:2, 4:2, 3:2, 2:2, 2:3, 2:4, 2:5, 2:6, 2:7, 2:8, 2:9, 2:10, 10:3, 9:3, 8:3, 7:3, 6:3, 5:3, 4:3, 3:3, 2:3, 3:2, 3:4, 3:5, 3:6, 3:7, 3:8, 3:9, 3:10, 10:4, 9:4, 8:4, 7:4, 6:4, 5:4, 4:4, 3:4, 2:4, 4:2, 4:3, 4:5, 4:6, 4:7, 4:8, 4:9, 4:10, 10:5, 9:5, 8:5, 7:5, 6:5, 5:5, 4:5, 3:5, 2:5, 5:2, 5:3, 5:4, 5:6, 5:7, 5:8, 5:9, 5:10, 10:6, 9:6, 8:6, 7:6, 6:6, 5:6, 4:6, 3:6, 2:6, 6:2, 6:3, 6:4, 6:5, 6:7, 6:8, 6:9, 6:10, 10:7, 9:7, 8:7, 7:7, 6:7, 5:7, 4:7, 3:7, 2:7, 7:2, 7:3, 7:4, 7:5, 7:6, 7:8, 7:9, 7:10, 10:8, 9:8, 8:8, 7:8, 6:8, 5:8, 4:8, 3:8, 2:8, 8:2, 8:3, 8:4, 8:5, 8:6, 8:7, 8:9, 1:10, 10:9, 9:9, 8:9, 7:9, 6:9, 5:9, 4:9, 3:9, 2:9, 9:2, 9:3, 9:4, 9:5, 9:6, 9:7, 9:8, or 9:10 by weight or by volume.

In one embodiment, the composition is a skin care composition for use in the treatment of a scalp disorder, comprising a fermentate mixture of at least two microorganisms selected from the group consisting of Yarrowia lipolytica and Bacillus velezensis, wherein said composition reduces and/or treats said scalp disorder, wherein said fermentate mixture comprises a fermentate supernatant of Yarrowia lipolytica (YSP) at about 0.01% to 10% by weight (or volume) and said fermentate supernatant of Bacillus velezensis (BSP) at about 0.01% to 10% by weight (or volume) relative to a total weight (or volume) of said skin care composition

In one aspect, the fermentate mixture comprises a fermentate supernatant of Yarrowia lipolytica (YSP) at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by weight relative to a total weight of said skin care composition

In one aspect, the fermentate mixture comprises a fermentate supernatant of Yarrowia lipolytica (YSP) at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by volume, relative to a total volume of said skin care composition

In one aspect, the fermentate mixture comprises a fermentate supernatant of Bacillus velezensis (BSP) at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by weight relative to a total weight of said skin care composition

In one aspect, the fermentate mixture comprises a fermentate supernatant of Bacillus velezensis (BSP) at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9% or up to 10% by volume, relative to a total volume of said skin care composition

The skin care composition of the present invention may further include one or more dermatologically or skin care acceptable component carrier.

The skin care compositions of the present invention include, but are not limited to, cosmetic products, aqueous solutions, emulsions, serums, jellies, patches, lotions, topical moisturizers, creams, pastes, balms, ointments, pomades, gels, liquids, sprays, foam, kits, or any one combinations thereof.

The skin care composition of the present invention includes, but is not limited to, a hair lotion, a shampoo, a hair conditioner, a detangler, a hair spray, a hair styling gel, a hair cream or gel, a styling lacquer, a hair setting lotion, a treating lotion, a dye composition, a hair-restructuring lotion, a permanent-waving composition, a lotion or gel for combating hair loss, an antiparasitic shampoo or a medicated shampoo, a scalp care product, or any one combination thereof.

In some embodiments, the skin care composition(s) of the invention may include isolated microorganism(s) suitable for use in the present invention and/or a cell lysate thereof, and/or fermentate thereof, and/or metabolite thereof, in combination with one or more dermatologically or skin care acceptable component carrier. The microorganism(s) suitable for use in the present invention can be live or non-replicating, e.g., inactivated, for example, by heat-treatment. The dosage may vary, but can range from the equivalent of about 10² to about 10¹² cfu/g, e.g., 1×10² cfu/g, 5×10² cfu/g. 1×10³ cfu/g. 5×10³ cfu/g, 1×10⁴ cfu/g. 5×10⁴ cfu/g, 1×10⁵ cfu/g, 5×10⁵ cfu/g, 1×10⁶ cfu/g, 5×10⁶ cfu/g, 1×10⁷ cfu/g, 5×10⁷ cfu/g, 1×10⁸ cfu/g, 5×10⁶ cfu/g, 1×10⁹ cfu/g, 5×10⁹ cfu/g, 1×10¹⁰ cfu/g, 5×10¹⁰ cfu/g, 1×10¹¹ cfu/g, 5×10¹¹ cfu/g, 1×10¹² cfu/g of dry weight.

In some embodiments, the microorganism(s) suitable for use in the present invention can be sterilized using conventional sterilization techniques before or after it is combined with the one or more dermatologically or skin care acceptable component

In one embodiment, the skin care composition is formulated for administration to the skin.

It will be further understood that the skin care composition for use in the present invention may further comprise one or more of probiotic bacteria in addition to the microorganism(s) suitable for use in the present invention, and/or a cell lysate thereof, and/or fermentate thereof, and/or metabolite thereof.

The skin care composition can comprise additional compounds selected from the group consisting of preservatives, pH adjusters, anti-oxidants and chelators.

Preservatives include but are not limited to parabens, sodium benzoate, potassium sorbate, phenyl ethyl alcohol, Lauryl ethyl arginate (LAE) and any combination thereof.

pH adjusters include but are not limited to weak acids, strong acids, any compound that can adjust the pH, such as but not limiting to citric acid, or any combination thereof.

In one embodiment, the skin care composition is formulated for topical administration to the skin/scalp.

The topical formulation (skin care composition) for use in the present invention may be in any form suitable for application to the scalp or skin surface, such as a cream, lotion, sprays, solution, gel, ointment, paste, plaster, paint, bioadhesive, suspensions or the like, and/or may be prepared so as to contain liposomes, micelles, and/or microspheres. Such a formulation may be used in combination with an occlusive overlayer so that moisture evaporating from the body surface is maintained within the formulation upon application to the body surface and thereafter.

Topical formulations include those in which the active ingredient(s) is (are) dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy) ethanol, propylene glycol, propylene glycol monolaurate, glycofurol or glycerol), oils (e.g. a mineral oil such as a liquid paraffin, natural or synthetic triglycerides, or silicone oils such as dimethicone). Depending, inter alia, upon the nature of the formulation as well as its intended use and site of application, the dermatological vehicle employed may contain one or more components (for example, when the formulation is an aqueous gel, components in addition to water) selected from the following list: a solubilizing agent or solvent (e.g. a ß-cyclodextrin, such as hydroxypropyl ß-cyclodextrin, or an alcohol or polyol such as ethanol, propylene glycol or glycerol); a thickening agent (e.g. hydroxyethylcellulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

The skin care composition of the present invention includes a liquid lotion (true solution) comprising water as a solvent and water soluble additives (solutes), such as but not limiting to an active, a fragrance, a color, a preservative, a pH adjuster, a chelating agent, or any one combination thereof.

The skin care composition of the present invention includes a dispersion such as an emulsion (such as, but not limited to the following: liquid in liquid [water in oil W/O, O/W, W/O/W], suspension [solid/liquid or liquid/solid], aerosol [liquid/gas or solid/gas], foam/mousse [gas/liquid or gas/emulsion, or gas/solid]). An example of an Oil in Water [O/W] emulsion includes, but is not limited to a combination of a water phase, an emulsifier, a fatty phase and an at least one additive. The water phase can comprise water, humectants and stabilizing agents [such as, but not limiting to, synthetic polymers, carbomers, natural polymers, xanthan gum, acacia gum, carragheenan, gellan, or any one combination thereof). Emulsifiers include, but are not limited to, anionic emulsifiers, cationic emulsifiers, non-ionic emulsifiers, amphoteric emulsifiers, silicone emulsifiers), auto emulsifying agents. Fatty phases (lipophilic ingredients) include, but are not limited to, waxes, butter, fatty esters, triglycerides, vegetal oil, mineral oil (parffinum), silicones, and thickeners/oil jellifying agents. Additives include, but are not limited to, preservative, fragrance (most often lipophilic), color, anti-oxidant, chelating agent, actives, pH adjuster (citric acid, lactic acid, AHA), neutralizers/strong basic agent like NaOH, Trimethylamine (for acrylic polymers to jellify) and powders.

The skin care composition of the present invention includes an aqueous gel comprising a water phase (including water, humectants, actives), a jellifying agent (such as but not limited to synthetic polymers, natural polymers, xanthan gum, acacia gum, carragheenan, gellan) and an additive (such as but not limited to fragrance, high HLB surfactant, color, actives, preservative system, pH adjuster, neutralizing agent, powders).

The skin care composition of the present invention includes a cleansing/surfactant system (such as but not limited to a shampoo, shower gel, micellar water) comprising a water phase (water, humectants), a surfactant, an additive (such as but not limited to fragrance, high HLB surfactant, color, actives, preservative system, pH adjuster, neutralizing agent, powders) and optionally a jellifying agent (such as but not limited to synthetic polymers, natural polymers, xanthan gum, acacia gum, carragheenan, gellan).

A dermatologically or skin care acceptable carrier may also be incorporated in the skin care composition of the present invention and may be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

The term “dermatologically acceptable” or “dermatologically acceptable carrier” or “skin care acceptable” or “skin care acceptable carrier” is used herein to refer to a compound or composition that may be incorporated into a dermatologically or skin care formulation without causing undesirable biological effects or unwanted interaction with other components of the formulation.

“Carriers” or “vehicles” as used herein refer to carrier materials suitable for incorporation in a topically applied composition. Carriers and vehicles useful herein include any such materials known in the art, which are nontoxic and do not interact with other components of the formulation in which it is contained in a deleterious manner.

The term “aqueous” refers to a formulation that contains water or that becomes water-containing following application to the skin or mucosal tissue.

Skin care products described herein may further comprise one or more dermatologically or skin care acceptable components known or otherwise effective for use skin care, provided that the optional components are physically and chemically compatible with the essential components described herein, or do not otherwise unduly impair product stability, aesthetics, or performance. Non-limiting examples of such optional components are disclosed in International Skin Care Ingredient Dictionary, Ninth Edition, 2002, and CTFA Skin Care Ingredient Handbook, Tenth Edition, 2004.

In one aspect, the dermatologically or skin care acceptable component is a dermatologically acceptable carrier comprising from about 10 wt. % to about 99.9 wt. %, alternatively from about 50 wt. % to about 95 wt. %, and alternatively from about 75 wt. % to about 95 wt. %, of a dermatologically acceptable carrier. Carriers suitable for use with the composition(s) may include, for example, those used in the formulation of mousses, tonics, gels, skin moisturizers and lotions. The carrier may comprise water; organic oils; silicones such as volatile silicones, amino or non-amino silicone gums or oils, and mixtures thereof; mineral oils; plant oils such as olive oil, castor oil, rapeseed oil, coconut oil, wheat germ oil, sweet almond oil, avocado oil, macadamia oil, apricot oil, safflower oil, candlenut oil, false flax oil, tamanu oil, lemon oil and mixtures thereof; waxes; and organic compounds such as C₂-C₁₀ alkanes, acetone, methyl ethyl ketone, volatile organic C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; hydroxypropylcellulose, and mixtures thereof. In one aspect, the carrier comprises water, fatty alcohols, volatile organic alcohols, and mixtures thereof. Other carriers can be formulated by those of ordinary skill in the art.

The skin care composition of the present invention described herein may further comprise from about 0.1% to about 10%, and alternatively from about 0.2% to about 5.0%, of a gelling agent to help provide the desired viscosity to the composition(s). Non-limiting examples of suitable optional gelling agents include crosslinked carboxylic acid polymers; unneutralized crosslinked carboxylic acid polymers; unneutralized modified crosslinked carboxylic acid polymers; crosslinked ethylene/maleic anhydride copolymers; unneutralized crosslinked ethylene/maleic anhydride copolymers (e.g., EMA 81 commercially available from Monsanto); unneutralized crosslinked alkyl ether/acrylate copolymers (e.g., SALCARE™ SC90 commercially available from Allied Colloids); unneutralized crosslinked copolymers of sodium polyacrylate, mineral oil, and PEG-1 trideceth-6 (e.g., SALCARE™ SC91 commercially available from Allied Colloids); unneutralized crosslinked copolymers of methyl vinyl ether and maleic anhydride (e.g., STABILEZE™ QM-PVM/MA copolymer commercially available from International Specialty Products); hydrophobically modified nonionic cellulose polymers; hydrophobically modified ethoxylate urethane polymers (e.g., UCARE™ Polyphobe Series of alkali swellable polymers commercially available from Union Carbide); and combinations thereof. In this context, the term “unneutralized” means that the optional polymer and copolymer gelling agent materials contain unneutralized acid monomers.

The dermatologically or skin care acceptable medium may contain a fatty substance in a proportion generally of from about 10 to about 90% by weight relative to the total weight of the product, where the fatty phase containing at least one liquid, solid or semi-solid fatty substance. The fatty substance includes, but is not limited to, oils, waxes, gums, and so-called pasty fatty substances. Alternatively, the products may be in the form of a stable dispersion such as a water-in-oil or oil-in-water emulsion. Additionally, the skin care products may contain one or more conventional skin care or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments (colorant agents).

The dermatologically acceptable carrier may be a moisturizer formulation containing at least one emulsifiers, at least one surfactant, or any combination thereof.

In one embodiment, the skin care product is a product comprising a first skin care composition and a second skin care composition, wherein the first skin care composition comprises an effective amount of a first active agent consisting of at least one microorganism of the genus Yarrowia and/or a fraction thereof, and/or a cell lysate thereof, and/or fermentate thereof, and/or metabolite thereof, wherein the second skin care composition comprises at least an effective amount of at least one second active agent (such as antidandruff active agents, skin conditioning agents, skin care active ingredient materials) for topical administration. Skin care compositions can further comprise skin care active ingredient materials including sun screen agents, moisturizers, humectants, benefiting agents skin, depositing agents such as surfactants, occlusive agents, moisture barriers, lubricants, emollients, anti-aging agents, antistatic agents, abrasive, antimicrobials, conditioners, exfoliants, fragrances, viscosifying agents, salts, lipids, phospholipids, vitamins, foam stabilizers, pH modifiers, preservatives, suspending agents, silicone oils, silicone derivatives, essential oils, oils, fats, fatty acids, fatty acid esters, fatty alcohols, waxes, polyols, hydrocarbons, and mixtures thereof.

In one aspect, the skin care composition of the present invention is a composition comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder; wherein the composition further comprises a moisturizing agent,

In one aspect, the skin care composition of the present invention is a composition comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder; wherein the composition further comprises at least an effective amount of at least one second active agent selected from antidandruff active agents,

Other ingredients that may be included in a skin care composition or skin care product include, without limitation, at least one active ingredient for the treatment or prevention of skin ailments, providing a skin care effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these, one or more natural moisturizing factors (such as betaine, xylitol, panthenol, ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example), glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

Any number of dermatologically acceptable materials commonly used in skin care products may also be incorporated into the present skin care products such as skin conditioning agents and skin colorants.

Skin conditioning agents as herein defined include, but are not limited to astringents, which tighten skin; exfoliants, which remove dead skin cells; emollients, which help maintain a smooth, soft, pliable appearance; humectants, which increase the water content of the top layer of skin; occlusives, which retard evaporation of water from the skin's surface; and miscellaneous compounds that enhance the appearance of dry or damaged skin or reduce flaking and restore suppleness. Skin conditioning agents are well known in the art, see for example Green et al. (WO01/07009), and are available commercially from various sources. Suitable examples of skin conditioning agents include, but are not limited to, lactobionic acid, gluconic acid, alpha-hydroxy acids, beta-hydroxy acids, polyols, hyaluronic acid, D,L-panthenol, polysalicylates, vitamin A palmitate, vitamin E acetate, glycerin, sorbitol, silicones, silicone derivatives, lanolin, natural oils, xylitol, fucose, rhamnose, and triglyceride esters. The skin conditioning agents may include polysalicylates, propylene glycol (CAS No. 57-55-6, Dow Chemical, Midland, MI), glycerin (CAS No. 56-81-5, Proctor & Gamble Co., Cincinnati, OH), glycolic acid (CAS No. 79-14-1, DuPont Co., Wilmington, DE), lactic acid (CAS No. 50-21-5, Alfa Aesar, Ward Hill, MA), malic acid (CAS No. 617-48-1, Alfa Aesar), citric acid (CAS No. 77-92-9, Alfa Aesar), tartaric acid (CAS NO. 133-37-9, Alfa Aesar), glucaric acid (CAS No. 87-73-0), galactaric acid (CAS No. 526-99-8), 3-hydroxyvaleric acid (CAS No. 10237-77-1), salicylic acid (CAS No. 69-72-7, Alfa Aesar), and 1,3 propanediol (CAS No. 504-63-2, DuPont Co., Wilmington, DE). Polysalicylates may be prepared by the method described by White et al. in U.S. Pat. No. 4,855,483, incorporated herein by reference. Glucaric acid may be synthesized using the method described by Merbouh et al. (Carbohydr. Res. 336:75-78 (2001). The 3-hydroxyvaleric acid may be prepared as described by Bramucci in published international patent application number WO 02/012530.

Skin care compositions can comprise skin care additives such as, but not limiting to, colorants/dyes, fragrances, actives, preservatives, pH adjusters, chelators, and anti-oxidants.

In one aspect, the skin care composition of the present invention is a composition comprising a first skin care composition and a second skin care composition, wherein the first skin care comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder; wherein the second skin care composition comprises at least an effective amount of at least one second active agent selected from antidandruff active agents, for topical administration. Examples of such antidandruff active agents include keratolytc agents such as salicylic acid and sulphur in its various forms, regulators of keratinization such as zinc pyrithione, a pyridinethione salt, a trihalocarbamide, triclosan, an azole compound, an antifungal polymer, allantoin, steroids such as topical corticosteroids, tar or polytar (coal tar), undecylenic acid, fumaric acid, an allylamine and mixtures thereof, ciclopirox, octopirox, piroctone olamine, clobetasol propionate, betamethasone valerate, tea tree oil, a mixed oil of thyme and catnip, topical antifungals such as selenium sulfide, imidazole (e.g. ketoconazole), hydroxypyridones (e.g. ciclopirox), naturopathic agents such as Melaleuca sp. oil, Aloe vera, and probiotic microorganisms. (Indian J. Dermatol, 2010 April-June; 55(2): 130-134).

Skin care compositions described herein can also be part of a kit for providing one or more skin care benefits such as, but not limiting to, a kit for preventing or reducing a dandruff condition

In one aspect the kit is a kit comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder and written instructions for administration to the subject in need.

### Methods for Microbial Treatment of a Scalp Disorder

The skin care compositions described herein can be used in methods for treating a scalp disorder.

Further provided herein are methods for treating a scalp disorder in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said scalp disorder.

In one aspect, the scalp disorder is selected from the group consisting of a dandruff condition of the scalp, unbalanced ecoflora of the scalp, discomfort of the scalp, tinea versicolor, dry skin, irritated skin, seborrheic dermatitis, and any one combination thereof.

In one embodiment, the method is a method for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition, wherein the skin care composition is administered to the subject's skin or scalp.

In one aspect, the microorganism and/or a cell lysate thereof, and/or fermentate thereof, and/or metabolite thereof, is a microorganism of the genus Yarrowia selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, or any one combination thereof.

In one aspect, the microorganism and/or a cell lysate thereof, and/or fermentate thereof, and/or metabolite thereof, is a Bacillus velezensis wherein the Bacillus velezensis, and/or a cell lysate thereof, and/or fermentate thereof, and/or metabolite thereof, is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

Methods for Treating and/or Reducing a Dandruff Condition.

The skin care compositions described herein can be used in methods method for treating and/or reducing a dandruff condition of the scalp in a subject in need thereof

Further provided herein are methods for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition.

In one aspect, the microorganism and/or a cell lysate thereof, and/or fermentate thereof, and/or metabolite thereof, is a microorganism of the genus Yarrowia selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, or any one combination thereof.

In one aspect, the microorganism and/or a cell lysate thereof, and/or fermentate thereof, and/or metabolite thereof, is a Bacillus velezensis wherein the Bacillus velezensis, and/or a cell lysate thereof, and/or fermentate thereof, and/or metabolite thereof, is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

In one aspect, the microorganism and/or a cell lysate thereof, and/or fermentate thereof, and/or metabolite thereof, is administered topically. In one embodiment, the method is A method for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition.

In one aspect, the microorganism and/or a fraction thereof, and/or a cell lysate thereof, and/or fermentate thereof, and/or metabolite thereof is administered topically.

In one embodiment, the method is a method for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition, wherein said composition is formulated in a single composition, and wherein the composition is administered to the subjects' skin or scalp.

In one embodiment, the method is a method for treating and/or reducing a dandruff condition of the scalp in a subject in need thereof, comprising administering at least two microorganisms selected from the group consisting of Yarrowia lipolytica and Bacillus velezensis, and/or a cell lysate thereof, and/or fermentate thereof, and/or metabolite thereof, to said subject, at least two microorganisms selected from the group consisting of Yarrowia lipolytica and Bacillus velezensis, and/or a cell lysate thereof, and/or fermentate thereof, and/or metabolite thereof, is formulated in a single composition, wherein the composition further comprises a compound selected from the group consisting of a skin care excipient, butyric acid, glucose, glycogen, magnesium ascorbyl phosphate, cetyl alcohol, dimethicone, isopropyl myristate, glycerol, propylene glycol, Quaternium-52, ethanol or any one combination thereof.

The skin care compositions described herein can be used in methods for treating a dandruff condition.

### General Definitions

The disclosures of all cited patent and non-patent literature are incorporated herein by reference in their entirety.

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values. As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

As used herein “administer” or “administering” is meant the action of introducing one or more microorganism (microbial strain), skin care composition(s), skin care formulation(s) and/or skin care product(s) to a subject in need for treatment of a scalp disorder.

Administering one or more microorganism (microbial strain), skin care composition(s), skin care formulation(s) and/or skin care product(s) to a subject includes applying or introducing one or more microorganism (microbial strain), skin care composition(s), skin care formulation(s) and/or skin care product(s) to a scalp, a skin surface, and to in-vitro or in-vivo skin cells.

As used herein, the term “biological contaminants” refers to one or more unwanted and/or pathogenic biological entities including, but not limited to, microorganisms, spores, viruses, prions, and mixtures thereof.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “embodiment” or “disclosure” is not meant to be limiting, but applies generally to any of the embodiments defined in the claims or described herein. These terms are used interchangeably herein.

As used herein, the term “excipient” refers to inactive substance used as a carrier for active ingredients, in a formulation. The excipient may be used to stabilize the active ingredient in a formulation, such as the storage stability of the active ingredient. Excipients are also sometimes used to bulk up formulations that contain active ingredients. An “active ingredient” includes a skin care benefit agent as described herein.

As used herein, the term “effective amount” refers to the amount sufficient to obtain the desired effect. A desired effect includes the prevention, reduction and/or treatment of a scalp disorder, such as the prevention, reduction and or treatment of dandruff condition.

As used herein, “prevent,” “preventing,” “prevention” and grammatical variations thereof refers to a method of partially or completely delaying or precluding the onset or recurrence of a disorder or condition (such as a scalp disorder) and/or one or more of its attendant symptoms or barring a subject from acquiring or reacquiring a disorder or condition or reducing a subject's risk of acquiring or reacquiring a disorder or condition or one or more of its attendant symptoms.

As used herein, the term “reducing”, “reduces” and grammatical variations thereof in relation to a particular trait, characteristic, feature, biological process, or phenomena refers to a decrease in the particular trait, characteristic, feature, biological process, or phenomena. The trait, characteristic, feature, biological process, or phenomena can be decreased by 5%, 10%, 15%, 20%, 25%, 30%, 35%, 40%, 45%, 50%, 55%, 60%, 65%, 70%, 75%, 80%, 85%, 90%, 95%, 100% or greater than 100%.

The terms “percent by weight”, “weight percentage (wt. %)” and “weight-weight percentage (% w/w)” are used interchangeably herein. Percent by weight refers to the percentage of a material on a mass basis as it is comprised in a composition, mixture, solution or product.

The term “16S rRNA” or “16S ribosomal RNA” means the rRNA constituting the small subunit of prokaryotic ribosomes. In bacteria, this sequence can be used to identify and characterize operational taxonomic units.

The term “ITS” or “Internal Transcribed Spacers” are regions within the ribosomal transcript that are excised and degraded during maturation. Their sequences can be used for phylogenetic analysis and/or identification of fungi or yeast.

The terms moisturizer, a lotion or a body lotion refer to a low to medium-viscosity emulsion of oil and water, most often oil-in-water but possibly water-in-oil with the primary benefit in a skin care application to hydrate the skin or to reduce its water loss. Nearly all moisturizers contain a combination of emollients, occlusives, and humectants. Emollients, which are mainly lipids and oils, hydrate and improve the appearance of the skin. A wide variety of suitable emollients is known and maybe used herein (International Skin Care Ingredient Dictionary and Handbook, eds. Wenninger and McEwen, pp. 1656-61, 1626, and 1654-55 (The Skin care, Toiletry, and Fragrance Assoc., Washington, D.C., 7th Edition, 1997) (referred to as “ICI Handbook”) contains numerous examples of suitable materials). Occlusives such as petrolatum, lanolin and bee wax reduce transepidermal water loss by creating hydrophobic barrier over the skin. Humectants such as glycerol and urea able to attract water from the external environment and enhance water absorption from the dermis into the epidermis. In addition, the moisturizer formulations may contain emulsifiers to maintain stability of emulsions, and use thickeners to achieve desired viscosity and skin feel. A wide variety of other ingredients such as fragrances, dyes, preservatives, therapeutic agents, proteins and stabilizing agents are commonly added for other consumer preferred attributes.

The term “percent (%) sequence identity” or “percent (%) sequence similarity,” as used herein with respect to a reference sequence is defined as the percentage of nucleotide residues in a candidate sequence that are identical to the residues in the reference polynucleotide sequence after optimal alignment of the sequences and introducing gaps, if necessary, to achieve the maximum percent sequence identity.

A microbial “strain” as used herein refers to a microorganism (such as a bacterium or fungus) which remains genetically unchanged when grown or multiplied. The multiplicity of identical microbes is included.

As used herein, the term a “biologically pure strain” means a strain containing no other microbial strains in quantities sufficient to interfere with replication of the strain or to be detectable by normal techniques. “Isolated” when used in connection with the organisms and cultures described herein includes not only a biologically pure strain, but also any culture of organisms which is grown or maintained other than as it is found in nature.

As used herein, the term “probiotic” or “probiotic microorganism” are used interchangeably herein and refer to a live microorganism (including bacteria or yeasts for example) which, when administered (topically or orally) in sufficient amounts, beneficially affects the host organism, i.e. by conferring one or more demonstrable benefits, such as a reduced dandruff condition, on the host organism. Whilst there are no lower or upper limits for probiotic use, it has been suggested that at least 106-1012, preferably at least 106-10¹⁰, preferably 108-10⁹, cfu as a daily dose will be effective to achieve the beneficial effects in a subject.

In one aspect the skin cells described herein are mammalian skin cells, such as human or animal skin cells.

The term “sequence identity” or “sequence similarity” as used herein, means that two polynucleotide sequences, a candidate sequence and a reference sequence, are identical (i.e. 100% sequence identity) or similar (i.e. on a nucleotide-by-nucleotide basis) over the length of the candidate sequence. In comparing a candidate sequence to a reference sequence, the candidate sequence may comprise additions or deletions (i.e. gaps) as compared to the reference sequence (which does not comprise additions or deletions) for optimal alignment of the two sequences. Optimal alignment of sequences for determining sequence identity may be conducted using the any number of publicly available local alignment algorithms known in the art such as ALIGN or Megalign (DNASTAR), or by inspection.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Non-limiting examples of compositions and methods disclosed herein include:

1. A skin care composition for use in the treatment of a scalp disorder, comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder.
1b. A skin care composition for use in the treatment of a scalp disorder, comprising an effective amount of at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder.
2. The skin care composition of embodiment 1, wherein the scalp disorder is selected from the group consisting of a dandruff condition of the scalp, unbalanced ecoflora of the scalp, discomfort of the scalp, tinea versicolor, dry skin, irritated skin, seborrheic dermatitis, and any one combination thereof.
3. The skin care composition of embodiment 1, wherein the fermentate is a fermentate supernatant mixture comprising a fermentate supernatant of Yarrowia lipolytica combined with a fermentate supernatant of Bacillus velezensis.
3b. The skin care composition of embodiment 1, wherein the effective amount of the fermentate of the at least two microorganisms selected from the group consisting of Yarrowia lipolytica and Bacillus velezensis, is an effective amount of a fermentate supernatant mixture comprising a fermentate supernatant of Yarrowia lipolytica combined with a fermentate supernatant of Bacillus velezensis.
3c. The skin care composition of embodiment 1, further comprising one or more antidandruff active agents.
4. The skin care composition of embodiment 3, wherein the fermentate supernatant of Yarrowia lipolytica (YSP) and the fermentate supernatant of Bacillus velezensis (BSP) are combined in a ratio of YSP:BSP from 10:1 to 1:10 by weight.
5. The skin care composition of embodiment 3, comprising said fermentate supernatant of Yarrowia lipolytica (YSP) at about 0.01% to 10% by weight and said fermentate supernatant of Bacillus velezensis (BSP at about 0.01% to 10% by weight relative to a total weight of said skin care composition.
6. The skin care composition of embodiment 1, wherein the composition reduces the growth of Malassezia species.
7. The skin care composition of embodiment 1, wherein the composition prevents or reduces biofilm formation of Malassezia species.
7b. The skin care composition of embodiment 1, wherein the composition removes biofilm of Malassezia species.
7c. The skin care composition of embodiment 6 or embodiment 7, wherein Malassezia species is selected from the group consisting of Malassezia restricta (M. restricta), Malassezia globosa (M. globosa), Malassezia furfur (M. furfur), Malassezia sympodialis (M. sympodialis), Malassezia phylotype 5 (M. phylotype 5), other uncharacterized Malassezia species, and any combination thereof.
8. The skin care composition of embodiment 1, further comprising a cosmetic or dermatological acceptable component.
8b. The skin care composition of embodiment 1, further comprising at least one additional compound selected from the group consisting of an excipient, a preservative, a pH adjuster.
9. The skin care composition of embodiment 1, wherein the Yarrowia lipolytica, and/or a cell lysate thereof, and/or fermentate thereof, and/or metabolite thereof, is selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, and any one combination thereof.
10. The skin care composition of embodiment 1, wherein the Bacillus velezensis, and/or a cell lysate thereof, and/or fermentate thereof, and/or metabolite thereof, is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
11. The skin care composition of embodiment 1, wherein the composition is selected from the group consisting of an aqueous solution, a hair lotion, a shampoo, a hair conditioner, a detangler, a hair spray, a hair styling gel, a hair cream or gel, a styling lacquer, a hair setting lotion, a treating lotion, a dye composition, a hair-restructuring lotion, a permanent-waving composition, a lotion or gel for combating hair loss, an antiparasitic shampoo or a medicated shampoo, a scalp care product, and any one combination thereof.
12. The skin care composition of embodiment 1, wherein the composition is formulated for topical application.
13. Use of the skin care composition of any preceding embodiment in the treatment of a scalp disorder
14. A method for treating a scalp disorder in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said scalp disorder.
14b. A method for treating a scalp disorder in a subject in need thereof, comprising administering an effective amount of a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said scalp disorder.
15. The method according to embodiment 14, wherein the scalp disorder is selected from the group consisting of a dandruff condition of the scalp, unbalanced ecoflora of the scalp, discomfort of the scalp, tinea versicolor, dry skin, irritated skin, seborrheic dermatitis, and any one combination thereof.
16. The method according to embodiment 15, wherein said skin care composition is administered topically.
17. A method for treating and/or reducing a dandruff condition of the scalp in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said dandruff condition.
18. The method of embodiment 17, wherein the skin care composition is administered to the subject's skin or scalp.
18 b. The method of embodiment 14 or 16, further comprising at least one or more dermatologically or skin care acceptable component.
19. The method of embodiment 14 or 17, wherein the Yarrowia lipolytica, and/or a cell lysate thereof, and/or fermentate thereof, and/or metabolite thereof, is selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, and any one combination thereof.
20. The method of embodiment 13 or 17, wherein the Bacillus velezensis, and/or a cell lysate thereof, and/or fermentate thereof, and/or metabolite thereof, is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.
21. The method of embodiment 13, wherein the fermentate, is a fermentate supernatant mixture comprising a fermentate supernatant of Yarrowia lipolytica combined with a fermentate supernatant of Bacillus velezensis.
22. The method of embodiment 21, wherein the fermentate supernatant mixture reduces the growth of Malassezia species and/or reduces biofilm formation of Malassezia species.
23. The method of embodiment 14 or 16, wherein the cell lysate thereof, and/or fermentate thereof, and/or metabolite thereof reduces the growth of Malassezia species, and/or reduces biofilm formation of Malassezia species, and/or prevents biofilm formation of Malassezia species.
24. A fermentate mixture comprising a fermentate of Yarrowia lipolytica combined with a fermentate from Bacillus velezensis for use in treatment of a scalp disorder.
25. A fermentate supernatant mixture comprising a fermentate supernatant of Yarrowia lipolytica combined with a fermentate supernatant of Bacillus velezensis for use in treatment of a scalp disorder.
26. The fermentate mixture use according to embodiment 24, wherein all or substantially all of the cells have been removed from the fermentate
27. The skin care composition of embodiment 1, wherein the cell lysate is a cell lysate mixture comprising a cell lysate from Yarrowia lipolytica combined with a cell lysate from Bacillus velezensis.
28. A skin care composition for use in the treatment of a scalp disorder in a subject in need thereof, comprising a cell lysate from Yarrowia lipolytica combined with a cell lysate from Bacillus velezensis, wherein said composition reduces and/or treats said scalp disorder.
29. The skin care composition of embodiment 1, comprising a combination of a cell lysate and a fermentate supernatant, wherein the cell lysate comprises a cell lysate of Yarrowia lipolytica and/or cell lysate of Bacillus velezensis, wherein the fermentate supernatant comprises a fermentate supernatant of Yarrowia lipolytica and/or fermentate supernatant of Bacillus velezensis.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “conc.” means concentration, “Trt” means treatment.

### Example 1

### Materials and Methods

### Yeast Strains

Yeasts were from ATCC (American Type Culture Collection), CBS (CBS-KNAW culture collection), or Danisco Global Culture Collection (DGCC). Strains used are M. globosa (CBS 7966, also known as ATCC MYA-4612), M. furfur (CBS 1878, also known as ATCC 14521), Y. lipolytica (ATCC 20362, ATCC 9773, DGCC9975).

### Bacillus Strains

Bacillus strains suitable for use in the present invention includes a bacterial strain having a 16S ribosomal RNA sequence displaying at least 97.0% sequence similarity to a 16S ribosomal RNA sequence of B. velezensis E04 (SEQ ID NO: 1) deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 (SEQ ID NO: 2) deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 (SEQ ID NO: 3) deposited at Westerdijk Fungal Biodiversity Institute (WFDB) under number CBS147471, and any one combination thereof.

### Growth Media

Cells were grown in modified Leeming & Notman (mLN) media containing 10 g/L bacteriological peptone, 2 g/L yeast extract, 8 g/L desiccated ox bile, 10 ml/L glycerol, 0.5 g/L glycerol monostearate, 5 ml/L Tween-60, 20 ml/L olive oil or palm oil. For synthetic growth media, 6.7 g/L yeast nitrogen base without amino acids, 6 g/L dipotassium phosphate, 4 g/L monopotassium phosphate, and different amounts of lipid as carbon sources and Tween-40, -60, or -80 as emulsifier. For solid media, 15 g/L agar was added. Medium was sterilized by autoclave at 110° C. for 20 minutes.

Cell Counting by qPCR

Quantitative PCR or qPCR was utilized to quantitate Yarrowia lipolytica and Malassezia globosa in individual and combined culture conditions at three timepoints; Day 1, Day 4 and Day 7. The qPCR assay used for the detection of all Malassezia globosa species was as described by Clavaud et al. (2013), PLOS One, 8:10. The primers/probe used were as follows; forward primer 1: 5′ CTAAATATCGGGGAGAGACCGA (SEQ ID NO:4), and reverse primer 2: 5′ GTACTTTTAACTCTCTTTCCAAAGTGCTT (SEQ ID NO:5), and the MGB Probe 1: FAM-TTCATCTTTCCCTCACGGTAC-MGB (SEQ ID NO:6). The qPCR assay for Yarrowia lipolytica targets the SNF1 gene and includes the following primers/probe; forward primer 3: 5′ ACACCATTCCCCCCTATCTGT (SEQ ID NO:7), reverse primer 4: 5′ TGACCACCAGCATCTGTTGAA (SEQ ID NO:8) and probe 2: 5′ 6FAM-TGCCGGCGCAAAACACCTG-TAMRA. ((SEQ ID NO:9) Genomic DNA from a representative strain of both Malassezia globosa and Yarrowia lipolytica were used to generate a standard curve for absolute quantitation.

One ml (1 ml) of culture for Yarrowia lipolytica and Malassezia globosa grown individually or in combination from each time point were centrifuged to pellet the yeast. Cell pellets were then utilized for genomic DNA extraction. The DNA was extracted using the Qiagen DNeasy PowerSoil Pro DNA kit (Qiagen™, Germantown, MD) as per the manufacturer's instructions. 1.5 μl of purified genomic DNA from each sample was combined with each of the following in two separate reactions for detection of Malassezia globosa or Yarrowia lipolytica; 10 μl ABI Universal TaqMan Mix w/o UNG, 0.2 μl 100 UM Forward and Reverse Primers, 0.05 μl TaqMan Probe and 8.05 μl Molecular Biology Grade water. Each sample was run in triplicate qPCR reactions on a Quantstudio 7 Instrument as follows: 10 min 95° C.+40 cycles (95° C. 15 sec+60° C. 60 sec). Fluorescent data was collected during amplification.

Linear regression analysis was used to establish standard curves for both Malassezia globosa and Yarrowia lipolytica based on known amounts of genomic DNA (gDNA) and their corresponding qPCR derived Ct values. These standard curves were then used to determine the copy number of each organism in samples from each of the three timepoints. Average values of the triplicate qPCR reactions were reported.

### Example 2

Isolation of Microbes from Soil Rhizosphere

A collection of microbes from rhizosphere soil of corn fields were used to generate cell free supernatant (fermentate). Growth media used for the isolation campaign include R2A, tryptic soy agar (TSA), and nutrient agar (NA). R2A medium consists of casein acid hydrolysate (0.5 g/L), dextrose (0.5 g/L), dipotassium phosphate (0.3 g/L), magnesium sulfate (0.024 g/L), proteose peptone (0.5 g/L), sodium pyruvate (0.3 g/L), soluble starch (0.5 g/L), and yeast extract (0.5 g/L). Each liter of tryptic soy agar contains 17.0 g tryptone, 3 g soytone, 2.5 g of glucose, 5 g of sodium chloride, 2.5 g of dipotassium phosphate, and 15 g of agar. NA medium has meat extract, 1 g/L, peptone, 5 g/L, sodium chloride, 5 g/L, yeast extract, 2 g/L and agar, 15 g/L.

Plant roots were excavated by piercing the soil with a shovel, and root-balls were cut to discard above ground plant mass. Bulk soil was removed by manually shaking. After shaking, soil closely associated with the root was collected in a wash pan with sterile water. Soil samples were suspended in sterile water. Serial dilutions up to 10-4 were plated to R2A, TSA, and NA media for isolation of a variety of microbes. Agar plates were incubated at 28° C. for 24-72 hours. Colonies were picked, grown in liquid medium, and stored at −80° C. in 25% glycerol. The phylogenetic identity of isolates was determined by sequencing the 16S region with primer set 8F (5′-AGA GTT TGA TYM TCC TGG CTC-3, SEQ ID NO: 10) and 1492R (5′-CGG TTA CCT TGT TAC GAC TT-3, SEQ ID NO: 11).

### Example 3

Malassezia Growth Inhibition Assays with Fermentate Supernatant

To generate fermentate supernatants (also referred to as cell free supernatants) from individual isolates described in Example 2, Bacillus velezensis strain E04 was revived from frozen stocks and grown in 1 mL TSB medium in deep-well microtiter plates. To prepare filtrate for microtiter plate assays, the candidate strains were sub-cultured by inoculating 25 μl of revived culture into 1100 μl fresh TSB in 96-deepwell microtiter plates and grown for three days at 28° C. in a chamber with 75% RH with shaking at 350 rpm. To collect fermentate supernatant, the fermentate (cell culture) was centrifuged at 4° C. 700 μl of the fermentate supernatant was removed and filtered through a 0.2 μm filter with a 96-well filter plate.

To generate different types of fermentates from Yarrowia lipolytica (Y. lipolytica) cultures, Y. lipolytica strain DGCC 9975 (Danisco Global Culture Collection 9975) or Y. lipolytica ATCC 20362 was grown in YPD (10 g/L yeast extract, 20 g/L peptone, 20 g/L glucose) media for 5 days with initial OD₆₀₀ of 0.1 at 32° C. with shaking at 250 rpm. Cell free Yarrowia culture supernatant (also referred to as fermentate supernatant) was prepared by filtering the Yarrowia cell culture (fermentate) though 0.22 μm filter membrane twice to remove cells and stored frozen at −20° C.

### Example 4

Flow Cytometry Analysis of M. globosa Growth Inhibition by Y. lipolytica and B. Velezensis Cell Free Culture Supernatant (Fermentate Supernatants)

Malassezia species (spp.) implicated in skin disorders do not have fatty acid synthase for lipid synthesis and therefore rely on sebum lipids from host for their growth (Xu et al., (2007) PNAS, 104: 18730). The growth medium for Malassezia species was a modified Leeming & Notman (mLN) media that contained 10 g/L bacteriological peptone, 2 g/L yeast extract, 4 g/L desiccated ox bile, 10 ml/L glycerol, 0.5 g/L glycerol monostearate, 5 ml/L Tween-60 and 20 ml/L palm oil.

Lipid emulsion by the presence of lipids and surfactants in the mLN media as well as cell clumping phenotype of Malassezia spp. make it difficult to monitor cell growth by optical density measurement or colony counting after spreading a culture aliquot on plates. To circumvent this issue, flow cytometry with cell staining dyes was used to quantitatively measure cell growth in lipid emulsion media. The flow cytometry analysis was conducted as follows to monitor the growth of M. globosa (CBS 7966) when challenged with Y. lipolytica cell free culture supernatants.

To monitor Malassezia globosa (M. globosa) growth inhibition by Y. lipolytica and/or B. velezensis cell free fermentate, three-day M. globosa culture inoculum was diluted 20 times in 1:1 mixture of fresh mLN media containing 20 ml/L palm oil and YPD. The diluted culture was then further diluted 2.5 times with sodium phosphate buffer (pH 6.0) to a final concentration of 0.2 M. One hundred (100) μl of Y. lipolytica or B. velezensis cell free culture supernatant, or one hundred (100) μl of 1:1 mixture of Y. lipolytica and B. velezensis cell free culture supernatant was added to 900 μl of the diluted M. globosa culture in 96-deep well plates (Biotix, San Diego, CA) and incubated at 32° C., shaken at 350 rpm with constant 85% humidity.

At different time points, 50 μl aliquot from each culture was subjected to mild sonication with amplitude of 5 and 1 sec on/off cycle for 45 sec using Qsonica Q700 (Qsonica, Newtown, CT). 20 μl of the sonicated cell culture was mixed with 50 μl phosphate-buffered saline, pH 7.4 (PBS), 10 μl diluted (1:1,000 in PBS) Cyto BC Green (Invitrogen), 10 μl diluted (1:1,000 in PBS) propidium iodide (Invitrogen), and 10 μl of diluted (1:200 in PBS) Concanavalin A (Invitrogen), and incubated for 20 min at room temperature. The samples were analyzed by flow cytometry using a Novocyte Quanteon (Acea). SytoBC, a cell permeable DNA/RNA intercalating dye was excited by the 488 laser and detected using a 530/30 bandwidth filter. Concanavalin A, a mannose binding lectin labeled with alexaFluor 640, was excited with the 637 nm laser and detected using a 660/20 bandwidth filter. Events with high mannose and high nucleic acid content were determined to be Malassezia cells versus events triggered by emulsion droplets which had comparatively low signal intensity in these channels. Malassezia cell counts were determined by gating on high sytoBC, high ConA events and recording the event/ul values for each sample.

Table 1 shows the flow cytometric analysis of M. globosa culture 4 days after the addition of various cell free culture supernatants from Y. lipolytica ATCC 20362, Y. lipolytica DGCC9975, and B. velezensis, or antifungal agent piroctone olamine (0.04% w/v). Percentages of M. globosa growth inhibition compared to YPD media control are shown in Table 1. The values are average of triplicate samples.

TABLE 1
Flow cytometric analysis of M. globosa culture 4 days
after the addition of various fermentate supernatants (cell
free culture supernatants) from Y. lipolytica strains
ATCC 20362 or DGCC9975, B. velezensis.

| | % M. globosa growth |
|---|---|
| | inhibition (+/−standard |
| | deviation) |
| Yarrowia ATCC 20362 | 58% (3%) |
| Yarrowia DGCC 9975 | 58% (6%) |
| B. velezensis | 62% (9%) |
| Yarrowia ATCC 20362 plus | 50% (4%) |
| B. velezensis (1:1 ratio) | |
| Yarrowia DGCC 9975 plus | 58% (16%) |
| B. velezensis (1:1 ratio) | |
| 0.04% Octopirox | 92% (1%) |

When 100 μl of 50:50 mixture by volume of fermentate supernatants from Y. lipolytica and B. velezensis were used, the percentage growth inhibition of M. globosa was comparable to that for 100 μl of fermentate supernatant from Y. lipolytica or B. velezensis by itself. Therefore, Y. lipolytica and B. velezensis fermentate supernatant showed an additive effect on M. globosa growth inhibition.

### Example 5

Additive M. globosa Growth Inhibition by Y. lipolytica and B. velezensis Cell Free Culture Supernatant (Cell Free Fermentate)

In this example, 20% (v/v) of fermentate supernatants (cell free supernatants) of Y. lipolytica and B. velezensis were used to confirm additive effect on M. globosa growth inhibition observed in Example 4 described herein. Two hundred (200) μl of Y. lipolytica or B. velezensis cell free culture supernatant, or two hundred (200) μl of 1:1 mixture of Y. lipolytica and B. velezensis cell free culture supernatant was added to 800 μl of the diluted M. globosa culture in 96-deep well plates (Biotix, San Diego, CA) and incubated at 32° C., shaken at 350 rpm with constant 85% humidity. Flow Cytometry Analysis of M. globosa Growth Inhibition as described in Example 4 was performed at different time points.

Table 2 shows the flow cytometric analysis of M. globosa culture 3 or 4 days after the addition of various cell free culture supernatants from Y. lipolytica ATCC 20362, Y. lipolytica DGCC9975, and B. velezensis, or antifungal agent piroctone olamine (0.05% w/v). Percentages of M. globosa growth inhibition compared to YPD media control are shown. The values are average of triplicate samples.

TABLE 2
Flow cytometric analysis of M. globosa culture 3 or 4 days after
the addition of various cell free culture supernatants from Y. lipolytica
strains ATCC 20362 or DGCC9975, B. velezensis.

| | Day 3 | Day 4 |
|---|---|---|
| | % M. globosa | % M. globosa |
| | growth inhibition | growth inhibition |
| | (+/−standard | (+/−standard |
| | deviation) | deviation) |
| Yarrowia ATCC 20362 | 71% (1%) | 73% (3%) |
| Yarrowia DGCC 9975 | 71% (6%) | 84% (3%) |
| B. velezensis | 84% (6%) | 84% (2%) |
| Yarrowia ATCC 20362 plus | 78% (10%) | 79% (3%) |
| B. velezensis (1:1 ratio) | | |
| Yarrowia DGCC 9975 plus | 85% (3%) | 88% (3%) |
| B. velezensis (1:1 ratio) | | |
| 0.04% Octopirox | 96% (1%) | 99% (1%) |

When 200 μl of 50:50 mixture (by volume) of fermentate supernatant from Y. lipolytica and B. velezensis was used, the percentage growth inhibition of M. globosa was comparable to that for 200 μl of fermentate supernatant from Y. lipolytica or B. velezensis by itself. Therefore, Y. lipolytica and B. velezensis fermentate supernatant showed an additive effect on M. globosa growth inhibition.

### Example 6

Removal of Biofilm of Malassezia Species by Combining Yarrowia and B. velezensis Fermentate Supernatants

Skin is a unique environment where microbes often exist as biofilms (Brandwein, et al., NPJ Biofilms Microbiomes 2:3, 2016). The biofilms can form on the epithelial surfaces of the skin or inside the follicles. In addition to cells, a biofilm consists of extracellular components such as exopolysaccharides, proteins, and DNA. This complex structure can be a physical and chemical barrier for certain compounds. But more importantly, the physiology of the microbes in the state of biofilm is very different than those in planktonic state. This is especially true for their ability to counter environmental stress and to resist various antimicrobial treatments, which provides remarkable therapeutic challenges (Koo, et al., Nature Reviews Microbiology 15:740-755, 2017).

Yeast Malassezia species isolated from both healthy and unhealthy skin have been shown to form biofilms in vitro (Angiolella, et al., Med Mycol. 0:1-7, 2020). These isolates of Malassezia globosa (M. globosa) can be highly adherent and/or hydrophobic as well as biofilm producers. Malassezia species in the form of biofilm have been shown to have a significant decrease in their susceptibility to antifungal agents (Figueredo, et al., Medical Mycology 8:863-867, 2013; Bumroogthai, et al., Medical Mycology 54:544-549, 2016). Biofilm adherence and hydrophobicity was suggested as virulence factor for Malassezia (Allen, et al., J. of Clinical & Experimental Dermatology Research 6:311, 2015; Angiolella, et al., Medical Mycology 56:110-116, 2018). Thus, strategies to remove Malassezia biofilm can be beneficial to treat various skin conditions caused by this group of organisms.

In this example, the efficacy of combining fermentate supernatants generated from Yarrowia and B. velezensis was evaluated. M. globosa (CBS 7966, also known as ATCC MYA-4612) or M. furfur (CBS 1878, also known as ATCC 14521) was used as pathogen target for the development of a biofilm assay. Y. lipolytica strains ATCC 20362 or ATCC 9773 were grown in YPD (10 g/L yeast extract, 20 g/L peptone, 20 g/L glucose), YPG (10 g/L yeast extract, 20 g/L peptone, 20 ml/L glycerol), mLN (10 g/L bacteriological peptone, 2 g/L yeast extract, 8 g/L desiccated ox bile, 10 ml/L glycerol, 0.5 g/L glycerol monostearate, 5 ml/L Tween-60) with 20 ml/L palm oil, or mLN with 20 ml/L olive oil for 5 days with initial OD600 of 0.1, at 32° C. with shaking at 250 rpm. Cell free Yarrowia culture supernatant (also referred to as cell free fermentate) was prepared by filtering the Yarrowia cell culture though 0.22 μm filter membrane twice to remove cells and stored at −20° C.

For B. velezensis strains, the growth medium can be tryptic soy broth (TSB) or malt extract broth (MEB). TSB consists of 17.0 g tryptone, 3 g of Soytone, 2.5 g of glucose, 5 g of sodium chloride, and 2.5 g of dipotassium phosphate per liter. MEB consists of 36 g of malt extract, 6 of peptone, and 2 ml of glycerol per liter. The broth pH was adjusted to 6.0.

The growth medium for Malassezia species was a modified Leeming & Notman (mLN) media that contained 10 g/L bacteriological peptone, 2 g/L yeast extract, 4 g/L desiccated ox bile, 10 ml/L glycerol, 0.5 g/L glycerol monostearate, 5 ml/L Tween-60 and 20 ml/L palm oil. After 3 days of incubation in a flask with 20 ml medium at 32° C. in a rotary shaker with a speed of 200 rpm, 10 μl culture was inoculated into wells of a polystyrene 96 well plate with 90 μl of mLN media using palm oil as the carbon source. The plate was incubated at 32° C. without shaking for 48 to 72 hours to allow growth of Malassezia strain as both suspending cells (planktonic cells) and as sessile biofilm cells attached to the wall of wells in the microtiter plate.

After the biofilm was formed, the growth medium and the unattached cells were removed, and wells were washed once with 1× phosphate buffered saline solution (PBS). PBS is a pH-adjusted blend of ultrapure-grade phosphate buffers and saline solutions which, when diluted to a 1× working concentration, contains 137 mM NaCl, 2.7 mM KCl, 8 mM Na₂HPO₄, and 2 mM KH₂PO₄. After washed, the biofilm plate was ready for treatments with 120 μl cell free supernatant (cell free fermentate).

The amount of cell free supernatant of Yarrowia or B. velezensis in the combination experiments can range anywhere from 5% to 95% each. For example, 50% each supernatant (60 μl Yarrowia supernatant and 60 μl of B. velezensis supernatant) can be mixed and used in the biofilm removal assay. For comparison, 120 μl of each supernatant alone was used. In addition, 120 μl of 1× PBS or medium was also used as control.

After the addition of fermentate or medium control, the biofilm plate was incubated at 32° C. for 20 min without shaking for the biofilm removal reaction. After incubation, the supernatant or PBS was removed. The amount of biofilm remaining in the wells were quantified by staining. The staining of biofilm was carried out by adding 120 μl of 0.1% crystal violet dissolved in water. The plate was incubated for 3 min at room temperature. After staining, 120 μl of 1×PBS was added to each well to remove the unbound dye. This process was repeated 1 more time. After wash, 120 μl of 70% ethanol was added to each well and the plate incubated for 5 min at room temperature to release the dye. The intensity of the dye was measured at 570 nm with a microtiter plate reader. The OD reading was used to quantify the amount of biofilm remained after each treatment. The wells treated with PBS were used as a reference to calculate the percentage of biofilm removal.

### Example 7

Prevention and reduction of biofilm formation of Malassezia species by Combining Yarrowia and B. velezensis fermentate

As stated previously, microbiome on skins surfaces including scalp is primarily present as biofilm communities. Just as important as the removal of pre-formed pathogenic biofilm, prevention and reduction of biofilm growth for Malassezia species is another strategy for the treatment of scalp disorders. In this example, the ability of Yarrowia and B. velezensis in combination to prevent or reduce the formation of Malassezia biofilm formation was evaluated.

As described in Example 5, Y. lipolytica strains ATCC 20362 or ATCC 9773 was grown in YPD (10 g/L yeast extract, 20 g/L peptone, 20 g/L glucose), YPG (10 g/L yeast extract, 20 g/L peptone, 20 ml/L glycerol), mLN (10 g/L bacteriological peptone, 2 g/L yeast extract, 8 g/L desiccated ox bile, 10 ml/L glycerol, 0.5 g/L glycerol monostearate, 5 ml/L Tween-60) with 20 ml/L palm oil, or mLN with 20 ml/L olive oil for 5 days with initial OD600 of 0.1, at 32° C. with shaking at 250 rpm. Cell free Yarrowia culture supernatant (also referred to as cell free fermentate) was prepared by filtering the Yarrowia cell culture though 0.22 μm filter membrane twice to remove cells and stored at −20° C.

As described in Example 5, for B. velezensis strains, the growth medium can be tryptic soy broth (TSB) or malt extract broth (MEB). TSB consists of 17.0 g tryptone, 3 g of Soytone, 2.5 g of glucose, 5 g of sodium chloride, and 2.5 g of dipotassium phosphate per liter. MEB consists of 36 g of malt extract, 6 of peptone, and 2 ml of glycerol per liter. The broth pH was adjusted to 6.0.

Either Malassezia globosa (M. globosa) ATCC MYA-4612 or Malassezia furfur (M. furfur) ATCC 14521 was used for biofilm growth assay. As described in Example 6, the strain was grown for 3 days in mLN media at 32° C. in a rotary shaker with a speed of 200 rpm as the starting culture. In a typical biofilm growth assay, 10 μl culture was inoculated into wells of a polystyrene 96 well plate containing 90 μl solution with a final OD (600 nm) of 0.25. This solution was consisted of MLN-palm oil media with or without fermentate. The final volume was 100 μl.

For the biofilm prevention and reduction, the amount of cell free supernatant of Yarrowia or B. velezensis in the combination can range anywhere from 5% to 95% each. For example, 50% each supernatant (10 μl Yarrowia supernatant and 10 μl of B. velezensis supernatant) can be mixed and used in the biofilm assay. For comparison, 20 μl of each supernatant alone was used. In addition, 20 μl of medium was used as control. The biofilm assay plate was incubated at 32° C. without shaking for 48 hours to allow biofilm growth of Malassezia strain.

After the biofilm was formed, the growth medium and the unattached cells were removed, and wells were washed once with 1× phosphate buffered saline solution (PBS). PBS is a pH-adjusted blend of phosphate buffers and saline solutions which, when diluted to a 1× working concentration, contains 137 mM NaCl, 2.7 mM KCl, 8 mM Na₂HPO₄, and 2 mM KH₂PO₄. The amount of biofilm remaining in the wells were quantified by staining with the addition of 120 μl of 0.1% crystal violet dissolved in water. The plate was incubated for 3 min at room temperature after the addition of the dye. After staining, 120 μl of 1×PBS was added to each well to remove the unbound dye. This process was repeated 1 more time. After wash, 120 μl of 70% ethanol was added to each well and the plate incubated for 5 min at room temperature to release the dye. The intensity of the dye was measured at 570 nm with a microtiter plate reader. The OD reading was used to quantify the amount of biofilm remained after each treatment. Result of the experiment wash shown below (Table 3).

TABLE 3
Biofilm Reduction and Prevention by fermentate of B. velezensis
and Yarrowia strains against M. furfur.

| Strains | Optical Density (OD) | % Inhibition |
|---|---|---|
| B. velezensis E04 (BV) | 0.2 | 96.3 |
| Yarrowia ATCC9773 | 3.6 | 43.1 |
| Yarrowia ATCC20362 | 1.6 | 74.9 |
| BV + ATCC9973 (50% | 1.3 | 79.7 |
| each by volume) | | |
| BV + ATCC20362 (50% | 1.5 | 75.6 |
| each by volume) | | |
| Medium control | 6.3 | 0.0 |

When the fermentate supernatant of B. velezensis was combined with that of Yarrowia ATCC 9773 at 50% each by volume, an additive activity was observed. An additive effect was also found when the fermentate supernatant of B. velezensis was combined with that of ATCC20362. This experiment showed that it is possible that fermentate supernatants from bacterial strain B. velezensis and yeast Yarrowia strains can be formulated together for the prevention and reduction of Malassezia biofilm.

### Example 8

Additive M. globosa Growth Inhibition by Y. lipolytica and B. velezensis Cell Lysates

In this example, cell lysates of Y. lipolytica and B. velezensis were used to test additive or synergistic effect on M. globosa growth inhibition.

To prepare B. velezensis lysate, cells were grown in mDix-TOBA media (description?) at 32 C with shaking at 200 rpm for 2 days. xx μL of the culture was harvested by centrifugation, and the cell pellet was washed once in x μl of PBS buffer. After centrifugation, the cell pellet was resuspended in 750 μL of PBS and lysed with glass-beads for 40 sec using FastPrep-24 high-speed benchtop homogenizer (MP Biomedical). After the homogenization, the sample was centrifuged, and the cell lysate was collected and filtered using 0.22 micron filter to remove any intact cells.

To prepare Yarrowia lysate, Yarrowia lipolytica ATCC20382 and DGCC9975 were grown in YPD at 32 C with shaking at 200 rpm for 5 days. 10 mL of the culture was harvested by centrifugation, and the cell pellet was washed once in 2 mL of PBS buffer. After centrifugation, the cell pellet was resuspended in 2 mL of PBS and lysed 3 times for 3 minutes with 1 min cooling at 4 C between the lysing steps, using FastPrep-24 high-speed benchtop homogenizer (MP Biomedical). After the homogenization, the sample was centrifuged, and the cell lysate was collected and filtered using 0.22 micron filter to remove any intact cells,

Two hundred (200) μl of Y. lipolytica or B. velezensis cell lysate, or two hundred (200) μl of 1:1 mixture of Y. lipolytica and B. velezensis lysate was added to 800 μl of the diluted M. globosa culture in 96-deep well plates (Biotix, San Diego, CA) and incubated at 32° C., shaken at 350 rpm with constant 75% humidity. Flow Cytometry Analysis of M. globosa Growth Inhibition as described in Example 4 was performed.

Table 4 shows the flow cytometric analysis of M. globosa culture 4 days after the addition of various cell lysates from Y. lipolytica ATCC 20362, Y. lipolytica DGCC9975, and B. velezensis, or antifungal agent piroctone olamine (0.06% w/v). Percentages of M. globosa growth inhibition compared to PBS buffer control are shown. The values are average of triplicate samples.

TABLE 4
Flow cytometric analysis of M. globosa culture 4 days after
the addition of the cell lysates from Y. lipolytica strains ATCC
20362 and DGCC9975, B. velezensis, or their mixture.

| | % M. globosa |
|---|---|
| | growth inhibition |
| PBS buffer control | 0% |
| Yarrowia ATCC 20362 | 13% |
| Yarrowia DGCC 9975 | 8% |
| B. velezensis | 7% |
| Yarrowia ATCC 20362 plus | 28% |
| B. velezensis (1:1 ratio) | |
| Yarrowia DGCC 9975 plus | 12% |
| B. velezensis (1:1 ratio) | |
| 0.06% Octopirox | 85% |

When 200 μl of 50:50 mixture of cell lysates from Y. lipolytica and B. velezensis was used, the percentage growth inhibition of M. globosa was stronger than that for 200 μl of cell lysates from Y. lipolytica or B. velezensis by itself. Therefore, Y. lipolytica and B. velezensis cell lysate showed an additive or synergistic effect on M. globosa growth inhibition.

1. A skin care composition for use in the treatment of a scalp disorder, comprising at least two microorganisms selected from the group consisting of Yarrowia lipolytica and Bacillus velezensis, and/or cell lysate thereof, and/or fermentate thereof, and/or metabolite thereof, wherein said composition reduces and/or treats said scalp disorder.

2. The skin care composition of claim 1, wherein the scalp disorder is selected from the group consisting of a dandruff condition of the scalp, unbalanced ecoflora of the scalp, discomfort of the scalp, tinea versicolor, dry skin, irritated skin, seborrheic dermatitis, and any one combination thereof.

3. The skin care composition of claim 1, wherein the fermentate is a fermentate supernatant mixture comprising a fermentate supernatant of Yarrowia lipolytica combined with a fermentate supernatant of Bacillus velezensis.

4. The skin care composition of claim 3, wherein the fermentate supernatant of Yarrowia lipolytica (YSP) and the fermentate supernatant of Bacillus velezensis (BSP) are combined in a ratio of YSP:BSP from 10:1 to 1:10 by weight or volume.

5. The skin care composition of claim 3, comprising said fermentate supernatant of Yarrowia lipolytica (YSP) at about 0.01% to 10% by weight and said fermentate supernatant of Bacillus velezensis (BSP at about 0.01% to 10% by weight relative to a total weight of said skin care composition.

6. The skin care composition of claim 1, wherein the composition reduces the growth of Malassezia species.

7. The skin care composition of claim 1, wherein the composition prevents or reduces biofilm formation of Malassezia species.

8. The skin care composition of claim 1, further comprising a cosmetic or dermatological acceptable component.

9. The skin care composition of claim 1, wherein the Yarrowia lipolytica, and/or a cell lysate thereof, and/or fermentate thereof, and/or metabolite thereof, is selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, and any one combination thereof.

10. The skin care composition of claim 1, wherein the Bacillus velezensis, and/or a cell lysate thereof, and/or fermentate thereof, and/or metabolite thereof, is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

11. The skin care composition of claim 1, wherein the composition is selected from the group consisting of an aqueous solution, a hair lotion, a shampoo, a hair conditioner, a detangler, a hair spray, a hair styling gel, a hair cream or gel, a styling lacquer, a hair setting lotion, a treating lotion, a dye composition, a hair-restructuring lotion, a permanent-waving composition, a lotion or gel for combating hair loss, an antiparasitic shampoo or a medicated shampoo, a scalp care product, and any one combination thereof.

12. The skin care composition of claim 1, wherein the composition is formulated for topical application.

13. (canceled)

14. A method for treating a scalp disorder in a subject in need thereof, comprising administering a skin care composition comprising at least two microorganisms selected from the group consisting of Yarrowia lipolytica and Bacillus velezensis, and/or a cell lysate thereof, and/or fermentate thereof, and/or metabolite thereof, to said subject, wherein said composition reduces and/or treats said scalp disorder.

15. The method according to claim 14, wherein the scalp disorder is selected from the group consisting of a dandruff condition of the scalp, unbalanced ecoflora of the scalp, discomfort of the scalp, tinea versicolor, dry skin, irritated skin, seborrheic dermatitis, and any one combination thereof.

16. The method according to claim 14, wherein said skin care composition is administered topically.

17. (canceled)

18. The method of claim 14, wherein the skin care composition is administered to the subject's skin or scalp.

19. The method of claim 14, wherein the Yarrowia lipolytica, and/or a cell lysate thereof, and/or fermentate thereof, and/or metabolite thereof, is selected from the group consisting of Yarrowia lipolytica ATCC 20362, Yarrowia lipolytica ATCC 9773, Yarrowia lipolytica DGCC 9975, Yarrowia lipolytica ATCC 18942, Yarrowia lipolytica ATCC 20177, Yarrowia lipolytica CBS2073, Yarrowia lipolytica Phaff #50-47, and any one combination thereof, and any one combination thereof.

20. The method of claim 14 or claim 17, wherein the Bacillus velezensis, and/or a cell lysate thereof, and/or fermentate thereof, and/or metabolite thereof, is selected from the group consisting of a bacterial strain having a 16S ribosomal RNA sequence displaying at least 97.0% sequence similarity to a 16S ribosomal RNA sequence of B. velezensis E04 deposited at Westerdijk Fungal Biodiversity Institute (WFDB) under number CBS147469, a bacterial strain having a 16S ribosomal RNA sequence displaying at least 97.0% sequence similarity to a 16S ribosomal RNA sequence of B. velezensis H02 deposited at Westerdijk Fungal Biodiversity Institute (WFDB) under number CBS 147473, a bacterial strain having a 16S ribosomal RNA sequence displaying at least 97.0% sequence similarity to a 16S ribosomal RNA sequence of B. velezensis F03 deposited at Westerdijk Fungal Biodiversity Institute (WFDB) under number CBS147471, and any one combination thereof.

21. The method of claim 14 or claim 17, wherein the fermentate is a fermentate supernatant mixture comprising a fermentate supernatant of Yarrowia lipolytica combined with a fermentate supernatant of Bacillus velezensis.

22. The method of claim 21, wherein the fermentate supernatant mixture reduces the growth of Malassezia species and/or reduces biofilm formation of Malassezia species.
